SEQUENCE LISTING (65 sequences)

SEQ ID NO: 1 - protein, 683 aa, Homo sapiens
MALFVRLLALALALALGPAATLAGPAKSPYQLVLQHSRLRGRQHGPNVCAVQKVIGTNRKYFTNCKQWYQRKICGKSTVISYECCPGYEKVPGEKGCPAALPLSNLYETLGVVGSTTTQLYTDRTEKLRPEMEGPGSFTIFAPSNEAWASLPAEVLDSLVSNVNIELLNALRYHMVGRRVLTDELKHGMTLTSMYQNSNIQIHHYPNGIVTVNCARLLKADHHATNGVVHLIDKVISTITNNIQQIIEIEDTFETLRAAVAASGLNTMLEGNGQYTLLAPTNEAFEKIPSETLNRILGDPEALRDLLNNHILKSAMCAEAIVAGLSVETLEGTTLEVGCSGDMLTINGKAIISNKDILATNGVIHYIDELLIPDSAKTLFELAAESDVSTAIDLFRQAGLGNHLSGSERLTLLAPLNSVFKDGTPPIDAHTRNLLRNHIIKDQLASKYLYHGQTLETLGGKKLRVFVYRNSLCIENSCIAAHDKRGRYGTLFTMDRVLTPPMGTVMDVLKGDNRFSMLVAAIQSAGLTETLNREGVYTVFAPTNEAFRALPPRERSRLLGDAKELANILKYHIGDEILVSGGIGALVRLKSLQGDKLEVSLKNNVVSVNKEPVAEPDIMATNGVVHVITNVLQPPANRPQERGDELADSALEIFKQASAFSRASQRSVRLAPVYQKLLERMKH

SEQ ID NO: 2 - protein, 103 aa, Homo sapiens
GPGSFTIFAPSNEAWASLPAEVLDSLVSNVNIELLNALRYHMVGRRVLTDELKHGMTLTSMYQNSNIQIHHYPNGIVTVNCARLLKADHHATNGVVHLIDKVI

SEQ ID NO: 3 - protein, 122 aa, Homo sapiens
TFETLRAAVAASGLNTMLEGNGQYTLLAPTNEAFEKIPSETLNRILGDPEALRDLLNNHILKSAMCAEAIVAGLSVETLEGTTLEVGCSGDMLTINGKAIISNKDILATNGVIHYIDELLIP

SEQ ID NO: 4 - protein, 112 aa, Homo sapiens
STAIDLFRQAGLGNHLSGSERLTLLAPLNSVFKDGTPPIDAHTRNLLRNHIIKDQLASKYLYHGQTLETLGGKKLRVFVYRNSLCIENSCIAAHDKRGRYGTLFTMDRVLTP

SEQ ID NO: 5 - protein, 133 aa, Homo sapiens
MGTVMDVLKGDNRFSMLVAAIQSAGLTETLNREGVYTVFAPTNEAFRALPPRERSRLLGDAKELANILKYHIGDEILVSGGIGALVRLKSLQGDKLEVSLKNNVVSVNKEPVAEPDIMATNGVVHVITNVLQP

SEQ ID NO: 6 - protein, 103 aa, mouse
GPGSFTIFAPSNEAWSSLPAEVLDSLVSNVNIELLNALRYHMVDRRVLTDELKHGMTLTSMYQNSNIQIHHYPNGIVTVNCARLLKADHHATNGVVHLIDKVI

SEQ ID NO: 7 - protein, 122 aa, mouse
TFETLRAAVAASGLNTVLEGDGQFTLLAPTNEAFEKIPAETLNRILGDPEALRDLLNNHILKSAMCAEAIVAGMSMETLGGTTLEVGCSGDKLTINGKAVISNKDILATNGVIHFIDELLIP

SEQ ID NO: 8 - protein, 112 aa, mouse
STAIDILKQAGLDTHLSGKEQLTFLAPLNSVFKDGVPRIDAQMKTLLLNHMVKEQLASKYLYSGQTLDTLGGKKLRVFVYRNSLCIENSCIAAHDKRGRFGTLFTMDRMLTP

SEQ ID NO: 9 - protein, 133 aa, mouse
MGTVMDVLKGDNRFSMLVAAIQSAGLMEILNREGVYTVFAPTNEAFQAMPPEELNKLLANAKELTNILKYHIGDEILVSGGIGALVRLKSLQGDKLEVSSKNNVVSVNKEPVAETDIMATNGVVYAINTVLQP

SEQ ID NO: 10 - protein, 98 aa, rat
HLSGKEQLTFLAPLNSVFKDGVPRIDGQMKTLLLNHMVKGQLASKYLYSGQTVDTLGGKKLRVFVYRNSLCIENSCIAAHDKKGRYGTLFTMDRMLTP

SEQ ID NO: 11 - protein, 111 aa, rat
MGTVMDVLKGDNRFSMLVAAIQSAGLMETLNREGVYTVFAPTNEAFQAMPPEELNKLLANAKELTNILKYHIGDEILVSGGIGALVRLKSLQGDKLEVSSKNNVVSVNKEP

SEQ ID NO: 12 - protein, 120 aa, Homo sapiens
TTTQRYSDASKLREEIEGKGSFTYFAPSNEAWDNLDSDIRRGLESNVNVELLNALHSHMINKRMLTKDLKNGMIIPSMYNNLGLFINHYPNGVVTVNCARIIHGNQIATNGVVHVIDRVL

SEQ ID NO: 13 - protein, 133 aa, Homo sapiens
TSIQDFIEAEDDLSSFRAAAITSDILEALGRDGHFTLFAPTNEAFEKLPRGVLERFMGDKVASEALMKYHILNTLQCSESIMGGAVFETLEGNTIEIGCDGDSITVNGIKMVNKKDIVTNNGVIHLIDQVLIP

SEQ ID NO: 14 - protein, 116 aa, Homo sapiens
KQQTTFTDLVAQLGLASALRPDGEYTLLAPVNNAFSDDTLSMVQRLLKLILQNHILKVKVGLNELYNGQILETIGGKQLRVFVYRTAVCIENSCMEKGSKQGRNGAIHIFREIIKP

SEQ ID NO: 15 - protein, 135 aa, Homo sapiens
EKSLHEKLKQDKRFSTFLSLLEAADLKELLTQPGDWTLFVPTNDAFKGMTSEEKEILIRDKNALQNIILYHLTPGVFIGKGFEPGVTNILKTTQGSKIFLKEVNDTLLVNELKSKESDIMTTNGVIHVVDKLLYP

SEQ ID NO: 16 - protein, 120 aa, mouse
TTTQHYSDVSKLREEIEGKGSYTYFAPSNEAWENLDSDIRRGLENNVNVELLNALHSHMVNKRMLTKDLKHGMVIPSMYNNLGLFINHYPNGVVTVNCARVIHGNQIATNGVVHVIDRVL

SEQ ID NO: 17 - protein, 133 aa, mouse
TSIQDFLEAEDDLSSFRAAAITSDLLESLGRDGHFTLFAPTNEAFEKLPRGVLERIMGDKVASEALMKYHILNTLQCSEAITGGAVFETMEGNTIEIGCEGDSISINGIKMVNKKDIVTKNGVIHLIDEVLIP

SEQ ID NO: 18 - protein, 116 aa, mouse
KQQTTFTDLVAQLGLASSLKPDGEYTLLAPVNNAFSDDTLSMDQRLLKLILQNHILKVKVGLSDLYNGQILETIGGKQLRVFVYRTAICIENSCMVRGSKQGRNGAIHIFREIIQP

SEQ ID NO: 19 - protein, 135 aa, mouse
EKSLHDKLRQDKRFSIFLSLLEAADLKDLLTQPGDWTLFAPTNDAFKGMTSEERELLIGDKNALQNIILYHLTPGVYIGKGFEPGVTNILKTTQGSKIYLKGVNETLLVNELKSKESDIMTTNGVIHVVDKLLYP

SEQ ID NO: 20 - protein, 120 aa, rat
TTTQHYSDVSKLREEIEGKGSYTYFAPSNEAWDNLDSDIRRGLENNVNVELLNALHSHMVNKRMLTKDLKHGMVIPSMYNNLGLFINHYPNGVVTVNCARVIHGNQIATNGVVHVIDRVL

SEQ ID NO: 21 - protein, 133 aa, rat
TSIQDFIEAEDELSSFRAAAITSDLLESLGRDGHFTLFAPTNEAFEKLPRGVLERIMGDKVASEALMKYHILNTLQCSEAITGGAVFETMEGNTIEIGCEGDSISINGIKMVNKKDIVTKNGVIHLIDEVLIP

SEQ ID NO: 22 - protein, 116 aa, rat; identical to SEQ ID NO: 18

SEQ ID NO: 23 - protein, 135 aa, rat
EKSLHEKLRQDKRFSIFLSLLEAADLKDLLTQPGDWTLFAPTNDAFKGMTNEEREILIGDKNALQNIILYHLTPGVYIGKGFEPGVTNILKTTQGSKIYVKGVNETLLVNELKSKESDIMTTNGVIHVVDKLLYP

SEQ ID NO: 24 - protein, 101 aa, Homo sapiens
TVLVPSVSSFSSRTMNASLAQQLCRQHIIAGQHILEDTRTQQTRRWWTLAGQEITVTFNQFTKYSYKYKDQPQQTFNIYKANNIAANGVFHVVTGLRWQAP

SEQ ID NO: 25 - protein, 104 aa, Homo sapiens
FTVFAPSNEAVDSLRDGRLIYLFTAGLSKLQELVRYHIYNHGQLTVEKLISKGRILTMANQVLAVNISEEGRILLGPEGVPLQRVDVMAANGVIHMLDGILLPP

SEQ ID NO: 26 - protein, 100 aa, Homo sapiens
TALVPSEAAVRQLSPEDRAFWLQPRTLPNLVRAHFLQGALFEEELARLGGQEVATLNPTTRWEIRNISGRVWVQNASVDVADLLATNGVLHILSQVLLPP

SEQ ID NO: 27 - protein, 93 aa, Homo sapiens
TIFVPTNRSLEAQGNSSHLDADTVRHHVVLGEALSMETLRKGGHRNSLLGPAHWIVFYNHSGQPEVNHVPLEGPMLEAPGRSLIGLSGVLTVG

SEQ ID NO: 28 - protein, 97 aa, Homo sapiens
TIFVPHADLMSNLSQDELARIRAHRQLVFRYHVVGCRRLRSEDLLEQGYATALSGHPLRFSEREGSIYLNDFARVVSSDHEAVNGILHFIDRVLLPP

SEQ ID NO: 29 - protein, 106 aa, Homo sapiens
TMLWPTDAAFRALPPDRQAWLYHEDHRDKLAAILRGHMIRNVEALASDLPNLGPLRTMHGTPISFSCSRTRPGELMVGEDDARIVQRHLPFEGGLAYGIDQLLEPP

SEQ ID NO: 30 - protein, 96 aa, Homo sapiens
TLFVPVNEGFVDNMTLSGPDLELHASNATLLSANASQGKLLPAHSGLSLIISDAGPDNSSWAPVAPGTVVVSRIIVWDIMAFNGIIHALASPLLAP

SEQ ID NO: 31 - protein, 103 aa, mouse
TVFAPSNEAVDSLRDGRLIYLFTAGLSKLQELVRYHIYNHGQLTVEKLISKGRVLTMANQVLTVNISEGGRILLGPGGIPVRRVDVPAANGVIHMLEGILLPP

SEQ ID NO: 32 - protein, 100 aa, mouse
TALVPSESAIRRLSLEDQAFWLQPKMLPELARAHFLQGAFSEEELARLNGQQVATLSATTRWQIHNISGKVWVQNATVDVPDLLATNGILHIVSQVLLPP

SEQ ID NO: 33 - protein, 93 aa, mouse
TIFVPTNHSLETQGNNSVLGIDTVRHHVILGEALSVEVLRKGGHRNSLLGPAHWLVFYNHSGQPEVNHMPLEGPLLEAPGSSLFGLSGILAVG

SEQ ID NO: 34 - protein, 97 aa, mouse
TVFVPHADLISNMSQDELARIRAHRQLVFRYHVVGCRKLWSQEMLDQGYITTLSGHTLRVSEREGSIYLNDFARVVSSDLEVVNGVLHFIDHVLLPP

SEQ ID NO: 35 - protein, 106 aa, mouse
TMLWPTDSALQALPPDRKNWLFHEDHRDKLAAILRGHMIRNIEALASDLPNLGQLRTMHGNTISFSCGLTRPGELIVGEDEAHIVQRHLTFEGGLAYGIDQLLEPP

SEQ ID NO: 36 - protein, 96 aa, mouse
TLFVPVNKGFVDNMTLSGPDLELHASNATFLSINASRGTLLPAHSGLSLFISDTGPDNTSLVPLAPGAVVVSHVIVWDIMAFNGIIHALASPLLMP

SEQ ID NO: 37 - protein, 103 aa, rat; identical to SEQ ID NO: 31

SEQ ID NO: 38 - protein, 100 aa, rat; identical to SEQ ID NO: 32

SEQ ID NO: 39 - protein, 93 aa, rat; identical to SEQ ID NO: 33

SEQ ID NO: 40 - protein, 97 aa, rat; identical to SEQ ID NO: 34

SEQ ID NO: 41 - protein, 106 aa, rat; identical to SEQ ID NO: 35

SEQ ID NO: 42 - protein, 96 aa, rat; identical to SEQ ID NO: 36

SEQ ID NO: 43 - protein, 103 aa, Homo sapiens
TVLLPTDKGLKGFNVNELLVDNKAAQYFVKLHIIAGQMNIEYMNNTDMFYTLTGKSGEIFNSDKDNQIKLKLHGGKKKVKIIQGDIIASNGLLHILDRAMDKL

SEQ ID NO: 44 - protein, 102 aa, Homo sapiens
TIFVPNNEALNNMKDGTLDYLLSPEGSRKLLELVRYHIVPFTQLEVATLISTPHIRSMANQLIQFNTTDNGQILANDVAMEEIEITAKNGRIYTLTGVLIPP

SEQ ID NO: 45 - protein, 101 aa, Homo sapiens
TVLVPSQQATEDMDQDEKSFWLSQSNIPALIKYHMLLGTYRVADLQTLSSSDMLATSLQGNFLHLAKVDGNITIEGASIVDGDNAATNGVIHIINKVLVPQ

SEQ ID NO: 46 - protein, 96 aa, Homo sapiens
TVFAPNNNAIENYIREKKVLSLEEDVLRYHVVLEEKLLKNDLHNGMHRETMLGFSYFLSFFLHNDQLYVNEAPINYTNVATDKGVIHGLGKVLEIQ

SEQ ID NO: 47 - protein, 97 aa, Homo sapiens
TVFAPLSAAFDEEARVKDWDKYGLMPQVLRYHVVACHQLLLENLKLISNATSLQGEPIVISVSQSTVYINNKAKIISSDIISTNGIVHIIDKLLSPK

SEQ ID NO: 48 - protein, 106 aa, Homo sapiens
TLFWPTDQALHALPAEQQDFLFNQDNKDKLKEYLKFHVIRDAKVLAVDLPTSTAWKTLQGSELSVKCGAGRDIGDLFLNGQTCRIVQRELLFDLGVAYGIDCLLID

SEQ ID NO: 49 - protein, 94 aa, Homo sapiens
TLFVPQNSGLGENETLSGRDIEHHLANVSMFFYNDLVNGTTLQTRLGSKLLITASQDPLQPTETRFVDGRAILQWDIFASNGIIHVISRPLKAP

SEQ ID NO: 50 - protein, 102 aa, mouse
TVLLPSDKGLKGVDVKELLMDKEAARYFVKLHIIAGQMSTEQMYNLDTFYTLTGKSGEIINKDKDNQLKLKLYGSKIVQIIQGNIVASNGLVHILDRAMDKI

SEQ ID NO: 51 - protein, 102 aa, mouse
TIFVPSNEALSNMTAGVLDYLLSPEGSRKLLELVRYHIVAFTQLEVATLVSTLHIRSMANQIITFNISSKGQILANNVAVDETEVAAKNGRIYTLTGVLIPP

SEQ ID NO: 52 - protein, 101 aa, mouse
TVLVPSLQAIKDMDQNEKSFWLSRNNIPALIKYHTLLGTYRVADLQTLPSSHMLATSLQGSFLRLDKADGNITIEGASFVDGDNAATNGVVHIINKVLIPQ

SEQ ID NO: 53 - protein, 96 aa, mouse
TVFVPNNEAIESYIREKKATSLKEDILQYHVVLGEKLLRNDLHNGMHRETMLGFSYLLAFFLHNDQLYVNEAPINYTNVATDKGVIHGLEKVLEIK

SEQ ID NO: 54 - protein, 97 aa, mouse
TVFVPSSDSFNSESKLKVWDKQGLMSQILRYHVVACQQLLLENLKVITSATTLQGEPISISVSQDTVLINKKAKVLSSDIISTNGVIHVIDTLLSPQ

SEQ ID NO: 55 - protein, 107 aa, mouse
TLFWPTDKALQALPQEQQDFLFNEDNKDKLKAYLKFHVIRDTMALASDLPRSASWKTLQGSELSVRCGTGSDVGELFLNGQMCRIIQRRLLFDGGVAYGIDCLLMDP

SEQ ID NO: 56 - protein, 93 aa, mouse
TLFVPQNSGLPKNKSLSGRDIEHHLTNVNVSFYDDLVNGTVLKTRLGSQLLITSSQDQLHQEARFVDGRAILQWDIIASNGVLHIISEPLKAP

SEQ ID NO: 57 - protein, 96 aa, rat
TVFVPNNEAIENYIREKKATSLKEDILRYHVVLGEKLLKNDLHNGMHRETMLGFSYLLAFFLRNDQLYVNEAPINYTNVATDKGVIHGLEKVLEIQ

SEQ ID NO: 58 - protein, 97 aa, rat
TVFAPLSSSFNHEPRIKDWDQQGLMSQVLRYHVVGCQQLLLDNLKVTTSATTLQGEPVSISVSQDTVFINNEAKVLSSDIISTNGVIHVIDKLLSPK

SEQ ID NO: 59 - protein, 107 aa, rat
TVFWPTDKALEALPPEQQDFLFNQDNKDKLKSYLKFHVIRDSKALASDLPRSASWKTLQGSELSVRCGTGSDIGELFLNEQMCRFIHRGLLFDVGVAYGIDCLLMNP

SEQ ID NO: 60 - protein, 94 aa, rat
TLFVPQNSGLPGNKSLSGRDIEHHLTNVNVSFYNDLVNGTFLRTMLGSQLLITFSQDQLHQETRFVDGRSILQWDIIAANGILHIISEPLRAPP

SEQ ID NO: 61 - protein, 103 aa, Homo sapiens; identical to SEQ ID NO: 2

SEQ ID NO: 62 - protein, 131 aa, Homo sapiens
NIQQIIEIEDTFETLRAAVAASGLNTMLEGNGQYTLLAPTNEAFEKIPSETLNRILGDPEALRDLLNNHILKSAMCAEAIVAGLSVETLEGTTLEVGCSGDMLTINGKAIISNKDILATNGVIHYIDELLI

SEQ ID NO: 63 - protein, 129 aa, Homo sapiens
PDSAKTLFELAAESDVSTAIDLFRQAGLGNHLSGSERLTLLAPLNSVFKDGTPPIDAHTRNLLRNHIIKDQLASKYLYHGQTLETLGGKKLRVFVYRNSLCIENSCIAAHDKRGRYGTLFTMDRVLTPP

SEQ ID NO: 64 - protein, 131 aa, Homo sapiens
MGTVMDVLKGDNRFSMLVAAIQSAGLTETLNREGVYTVFAPTNEAFRALPPRERSRLLGDAKELANILKYHIGDEILVSGGIGALVRLKSLQGDKLEVSLKNNVVSVNKEPVAEPDIMATNGVVHVITNVL

SEQ ID NO: 65 - DNA, 420 bp, Homo sapiens
ctgacccccccaatggggactgtcatggatgtcctgaagggagacaatcgctttagcatgctggtagctgccatccagtctgcaggactgacggagaccctcaaccgggaaggagtctacacagtctttgctcccacaaatgaagccttccgagccctgccaccaagagaacggagcagactcttgggagatgccaaggaacttgccaacatcctgaaataccacattggtgatgaaatcctggttagcggaggcatcggggccctggtgcggctaaagtctctccaaggtgacaagctggaagtcagcttgaaaaacaatgtggtgagtgtcaacaaggagcctgttgccgagcctgacatcatggccacaaatggcgtggtccatgtcatcaccaatgttctgcagcctccagccaac

(19) United States
(12) Patent Application Publication
(10) Pub. No.: US 2008/0274955 A1
(43) Pub. Date: Nov. 6, 2008

(54) Novel Use of a Polypeptide Comprising Fas-1 Domain

(75) Inventors: Ju-Ock Nam, Daegu (KR); Jung-Eun Kim, Daegu (KR); Ha-Won Jeong, Daegu (KR); Sung-Jin Lee, Daegu (KR); Byung-Heon Lee, Daegu (KR); Je-Yong Choi, Daegu (KR); Rang-Woon Park, Daegu (KR); Jae-Yong Park, Daegu (KR); In-San Kim, Daegu (KR)

Correspondence Address: BUCHANAN, INGERSOLL & ROONEY PC, POST OFFICE BOX 1404, ALEXANDRIA, VA, 22313-1404, US

(73) Assignee: KYUNGPOOK NATIONAL UNIVERSITY INDUSTRY ACADEMIC COOPERATION FOUNDATION, Daegu (KR)

(21) Appl. No.: 11/578,463

(22) PCT Filed: Apr. 13, 2004
(86) PCT No.: PCT/KR04/00851

§ 371 Date: Oct. 13, 2006

(51) Int. Cl. A61K 38/16; C07K 14/00

(52) U.S. Cl. 514/12; 530/350

(57) ABSTRACT

The present invention relates to the novel use of a polypeptide comprising a fas-1 domain. More particularly, the invention relates to a method for the inhibition of the adhesion, migration and/or proliferation of endothelial cells, and/or for the inhibition of angiogenesis, using the polypeptide comprising the fas-1 domain, or functional equivalents thereof. Furthermore, the invention provides a method for treating or preventing angiogenesis-related diseases, using the polypeptide.

### TECHNICAL FIELD

The present invention relates to a polypeptide having an angiogenesis-inhibitory effect, and more particularly, to the anti-angiogenic use of a polypeptide comprising a fas-1 domain.

### BACKGROUND ART

Angiogenesis is defined as the formation of new capillary blood vessels from preexisting micro-vessels. Normal angiogenesis occurs during embryogenic development, tissue remodeling, organ growth, wound healing and female reproductive cycles (corpus luteum development) under tight physiological regulation (Folkman and Cotran, Int. Rev. Exp. Patho., 16:207-248, 1976). Generally, angiogenesis involves the proteolysis of the blood vessel basement membrane by proteases, followed by the migration, proliferation and differentiation of endothelial cells to form tubules and eventually the regeneration of new blood vessels.

Unregulated and abnormal angiogenesis may lead to various diseases. Examples of angiogenesis-related diseases that occur in pathological conditions include various cancers (tumors); vascular diseases such as vascular malformation, arteriosclerosis, vascular adhesions, and edematous sclerosis; ocular diseases such as corneal graft neovascularization, neovascular glaucoma, diabetic retinopathy, angiogenic corneal disease, macular degeneration, pterygium, retinal degeneration, retrolental fibroplasia and granular conjunctivitis; inflammatory diseases such as rheumatoid arthritis, systemic Lupus erythematosus and thyroiditis; and dermatological diseases such as psoriasis, capillarectasia, pyogenic granuloma, seborrheic dermatitis and acne (U.S. Pat. No. 5,994,292; Korean Patent Application Laid-Open No. 2001-66967; D'Amato R. J. et al., Ophtahlmol., 102:1261-1262, 1995; Arbiser J. L. J. Am. Acad. Derm., 34(3):486-497, 1996; O'Brien K. D. et al., Circulation, 93(4):672-682, 1996; Hanahan D. et al., Cell, 86:353-364, 1996).

Thus, studies on the mechanism of angiogenesis and the discovery of substances capable of inhibiting angiogenesis are of significant importance in the prevention and treatment of various diseases, including cancer. Current studies on the inhibition of angiogenesis are being conducted on target genes by various strategies, including a strategy of administering a competitive substance to inhibit the action of VEGF and bFGF (basic fibroblast growth factor), which are known as potent inducers of angiogenesis, and a strategy of regulating the expression of integrin in vascular endothelial cells to inhibit the metastasis of the cancel cells. Regarding the relationship of angiogenesis with cancer, studies on the correlation between vascular absorption and angiogenesis induced by cancer cells and on proteins that induce angiogenesis are being conducted but are still large incomplete. Studies on angiogenic inhibition are applicable to the diagnosis, treatment and/or prevention of a variety of angiogenesis-related diseases, and thus, there is a continued need for research and development regarding angiogenesis.

Meanwhile, fas-1 domains are highly conserved sequences found in some secretory and membrane proteins of several species including mammals, insects, sea urchins, plants, yeast, and bacteria (Kawamoto T., et al., Biochem. Biophys. Acta., 288-292, 1998). Each of the fas-1 domains consists of 110-140 amino acids and comprises two highly conserved branches of about 10 amino acids (H1 and H2) (Kawamoto T., et al., Biochim. Biophys. Acta., 288-292, 1998). Examples of proteins comprising the fas-1 domains include βig-h3, periostin, fasciclin I, sea urchin HLC-2, algal-CAM and mycobacterium MPB70 (Huber, O. et al., EMBO J., 4212-4222, 1994; Matumoto, S. et al., J. Immunol., 281-287, 1995; Takeshita, S. et al., Biochem. J., 271-278, 1993; and Wang, W. C. et al., J. Biol. Chem., 1448-1455, 1993). Of such proteins, βig-h3, periostin and fasciclin I have four fas-1 domains, HLC-2 has two fas-1 domains, and MPB70 has only one fas-1 domain. Although the biological functions of the proteins containing the fas-1 domains were not clearly revealed, several proteins were reported to act as a cell adhesion molecule. Of them, βig-h3 was reported to mediate the adhesion of fibroblasts and epithelial cells, and periostin the adhesion of osteoblasts, and fasciclin I the adhesion of nerve cells (LeBaron, R. G. et al., J. Invest. Dermatol., 844-849, 1995; Horinchi, K. et al., J. Bone Miner. Res., 1239-1249, 1999; and Wang, W. C. et al., J. Biol. Chem., 1448-1455, 1993). Also, algal-CAM is known to be a cell adhesion molecule in embryos of the alga Volvox (Huber, O. et al., EMBO J., 4212-4222, 1994).

However, the specific physiological functions, particularly angiogenesis-related functions, of the fas-1 domains, are not yet identified.

### DISCLOSURE OF THE INVENTION

Accordingly, the present inventors have conducted extensive studies on the physiological functions of the fas-1 domains, and consequently, found that the fas-1 domains show a potent anti-angiogenic effect, thereby completing the present invention.

An object of the present invention is to provide the novel use of an isolated polypeptide comprising a fas-1 domain.

To achieve the above object, in one aspect, the present invention provides a method for inhibiting the adhesion, migration and/or proliferation of endothelial cells, the method comprising administering to a subject in need thereof an effective amount of an isolated polypeptide comprising a fas-1 domain.

In another aspect, the present invention provides a method for inducing the apoptosis of endothelial cells, the method comprising administering to a subject in need thereof an effective amount of an isolated polypeptide comprising a fas-1 domain.

In still another aspect, the present invention provides a method for inhibiting angiogenesis, which comprises administering to a subject in need thereof an effective amount of an isolated polypeptide comprising a fas-1 domain.

In still another aspect, the present invention provides a pharmaceutical composition for the inhibition of angiogenesis or for the treatment or prevention of angiogenesis-related diseases, the composition comprising as an active ingredient an isolated polypeptide comprising a fas-1 domain.

In yet another aspect, the present invention provides the use of an isolated polypeptide comprising a fas-1 domain, for the preparation of a pharmaceutical agent for inhibiting the adhesion, migration and/or proliferation of endothelial cells.

In yet another aspect, the present invention provides the use of an isolated polypeptide comprising a fas-1 domain, for the preparation of a pharmaceutical agent for inducing the apoptosis of endothelial apoptosis.

In another further aspect, the present invention provides the use of an isolated polypeptide comprising a fas-1 domain, for the preparation of an agent for inhibiting angiogenesis or an agent for treating or preventing angiogenesis-related diseases.

As used herein, the term “effective amount” is defined as an amount at which one or more effects selected from the group consisting of the inhibition of endothelial cell migration, adhesion and/or proliferation, the inhibition of angiogenesis, and the induction of endothelial cell apoptosis are shown.

As used herein, the term “subject” means animals, including mammals, particularly human beings. The subject may preferably be a patient who requires treatment.

Hereinafter, the present invention will be described in detail.

In the isolated polypeptide comprising the fas-1 domain according to the present invention, the fas-1 domain may be derived from mammals, and preferably any one selected from the group consisting of human beings, rats and mice. In the present invention, fas-1 domains derived from all proteins known to contain the fas-1 domain may all be used. Thus, fas-1 domains, which are searched through protein sequence databases known in the art, for example NCBI Entrez (http://www.ncbi.nlm.nih.gov/Entrez/), EMBL-EBI (http://www.ebi.ac.uk/) or SMART (http://smart.emblheidelberg.de/), may all be used in the present invention. Particularly, the fas-1 domain used in the present invention preferably contains an YH motif. As used herein, the term “YH motif” is defined as an amino acid sequence comprising tyrosine-histidine (Y-H) or asparagine-histidine (N-H) residues highly conserved in the fas-1 domains of a βig-h3 protein, and several hydrophobic amino acid residues (e.g., leucine and isoleucine) adjacent to the conserved residues (Kim, J.-E. et al., J. Biol. Chem., 277:46159-46465, 2002). The YH motif is also highly conserved in fas-1 domains derived from other proteins, in addition to the βig-h3 protein (see FIG. 1). Concretely, the YH motif may be: (a) an isolated peptide consisting of at least 18 amino acids, comprising tyrosine-histidine (Y-H) or asparagine-histidine (N-H), and at least three hydrophobic amino acids with bulky side chains; or (b) a mutant or derivative of the peptide (a), in which the tyrosine-histidine (Y-H) or asparagine-histidine (N-H) in the peptide (a) were substituted with amino acids selected from the group consisting of serine-histidine (S-H), histidine-histidine (H-H), phenylalanine-histidine (F-H), threonine-histidine (T-H), tyrosine-asparagine (Y-N) and alanine-alanine (A-A).

In the present invention, the fas-1 domain derived from one protein selected from βig-h3, periostin, stabilin-I and stabilin-II may preferably be used. More preferably, the following fas-1 domain may be used: the fas-1 domains of human βig-h3, represented by SEQ ID NO: 2 to SEQ ID NO: 5; the fas-1 domains of mouse βig-h3, represented by SEQ ID NO: 6 to SEQ ID NO: 9; the fas-1 domain of rat βig-h3, represented by SEQ ID NO: 10 or 11; the fas-1 domains of human periostin, represented by SEQ ID NO: 12 to SEQ ID NO: 15; the fas-1 domains of mouse periostin, represented by SEQ ID NO: 16 to SEQ ID NO: 19; the fas-1 domains of rat periostin, represented by SEQ ID NO: 20 to SEQ ID NO: 23; the fas-1 domains of human stabilin-I, represented by SEQ ID NO: 24 to SEQ ID NO: 30; the fas-1 domains of mouse stabilin-I, represented by SEQ ID NO: 31 to SEQ ID NO: 36; the fas-1 domains of rat stabilin-I, represented by SEQ ID NO: 37 to SEQ ID NO: 42; the fas-1 domains of human stabilin-II, represented by SEQ ID NO: 43 to SEQ ID NO: 49; the fas-1 domains of mouse stabilin-II, represented by SEQ ID NO: 50 to SEQ ID NO: 56; and the fas-1 domains of rat stabilin-II, represented by SEQ ID NO: 57 to SEQ ID NO: 60. Most preferably, the fas-1 domains of human βig-h3, represented by SEQ ID NO: 2 to SEQ ID NO: 5, or the fas-1 domain of human stabilin-II, represented by SEQ ID NO: 45 or 49, may be used in the present invention. Furthermore, polypeptides represented by SEQ ID NO 61 to SEQ ID NO: 64, which contain the fas-1 domains of human βig-h3 represented by SEQ ID NO: 2 to SEQ ID NO: 5, may also be used in the present invention. In addition, fas-1 domains derived from fas-1 domain-comprising proteins may be used alone or in a combination of two or more.

Moreover, functional equivalents or salts of the fas-1 domain are within the scope of the inventive polypeptide. As used herein, the term “functional equivalents” is defined as polypeptides showing substantially the same physiological activity as that of the fas-1 domain according to the invention. The polypeptides having the same or similar structure as the inventive polypeptide as well as amino acid sequences are within the scope of the present invention insofar as they show substantially the same physiological activity as that of the inventive polypeptide. As used herein, the term “substantially the same physiological activity” is defined as one or more activities selected from the group consisting of the inhibition of endothelial cell adhesion, migration and/or proliferation, the inhibition of endothelial cell apoptosis, the inhibition of angiogenesis and/or the inhibition of tumor growth, which result from the interaction between the inventive polypeptide and the αvβ3 integrin of endothelial cells.

Examples of the functional equivalents include certain amino acid sequence variants in which parts or the whole of a fas-1 domain was substituted, or parts of the amino acids of the fas-1 domain were deleted or added. Substitutions of the amino acids are preferably conservative substitutions. Examples of conservative substitutions of naturally occurring amino acids are as follows: aliphatic amino acids (Gly, Ala, Pro), hydrophobic amino acids (Ile, Leu, Val), aromatic amino acids (Phe, Tyr, Trp), acidic amino acids (Asp, Glu), basic amino acids (His, Lys, Arg, Gln, Asn) and sulfur-containing amino acids (Cys, Met). Deletions of the amino acids are preferably located at regions that are not directly involved in the physiological activity of the fas-1 domains.

Furthermore, the definition of the functional equivalents according to the present invention also encompasses polypeptide derivatives in which some of the chemical structure of the inventive polypeptide was modified while maintaining the backbone and physiological activity of the inventive polypeptide. Examples thereof include structural modifications to modify the stability, storage, volatility and solubility of the inventive polypeptide.

The inventive polypeptide may be easily prepared by a chemical synthesis method known in the art (Creighton, Proteins; Structures and Molecular Principles, W. H. Freeman and Co., NY, 1983). Typical methods include but are not limited to liquid or solid state synthesis, fragment condensation, and F-MOC or T-BOC chemistry (Chemical Approaches to the Synthesis of Peptides and Proteins, Williams et al., Eds., CRC Press, Boca Raton Fla., 1997; A Practical Approach, Atherton & Sheppard, Eds., IRL Press, Oxford, England, 1989).

Moreover, the inventive polypeptide may be prepared by a genetic engineering method. For this purpose, a DNA sequence encoding the inventive polypeptide is first constructed according to a conventional method. The DNA sequence can be constructed by PCR-amplification with suitable primers. Alternately, the DNA sequence may also be synthesized by any standard method known in the art, for example, using an automated DNA synthesis system (sold from Biosearch or Applied Biosystems). The constructed DNA sequence is inserted into a vector containing one or more expression control sequences (e.g., promoter and enhancer, etc.), which are operatively linked to the DNA sequence to control the expression of the DNA sequence. Host cells are then transformed with the resulting recombinant expression vector. The transformed cells are incubated in a suitable medium under the condition to express the DNA sequence, and a substantially pure polypeptide encoded by the DNA sequence is recovered. The polypeptide recovery may be performed by a method known in the art (e.g., chromatography). As used herein, the term “substantially pure polypeptide” means that the inventive polypeptide does not substantially contain any proteins derived from host cells. For the genetic engineering method for synthesizing the inventive polypeptide, reference may be made to the following publications: Maniatis et al., Molecular Cloning; A laboratory Manual, Cold Spring Harbor laboratory, 1982; Sambrook et al., supra; Gene Expression Technology, Method in Enzymology, Genetics and Molecular Biology, Method in Enzymology, Guthrie & Fink (eds.), Academic Press, San Diego, Calif., 1991; and Hitzeman et al., J. Biol. Chem., 255:12073-12080, 1990.

Previously, the present inventors reported that the fas-1 domains of βig-h3 have not only an α3β1 integrin-interacting motif that induces the adhesion of epithelial cells (Kim, J.-E. et al., J. Biol. Chem., 275:30907-30915, 2000), but also an αvβ5 integrin-interacting motif that mediates the adhesion of fibroblasts (Kim, J.-E. et al., J. Biol. Chem., 277:46159-46165, 2002). Also, the present inventors found previously that an YH motif that is conserved in the fas-1 domains of βig-h3 inhibits endothelial cell adhesion and migration, and angiogenesis (Nam, J. O. et al., J. Biol. Chem., 278:25902-25909, 2003).

Thus, the present inventors tested whether each of the fas-1 domains of βig-h3 shows endothelial cell adhesion activity. Also, an integrin receptor that is involved in the adhesion of endothelial cells to βig-h3 was identified (see Examples 1 to 3).

The results showed that βig-h3 and its fas-1 domains mediated the adhesion of endothelial cells at almost equal activity (see FIG. 2), and that the αvβ3 integrin is involved in the adhesion of endothelial cells by interaction with βig-h3 (see FIGS. 3a to 3d).

Thereafter, the present inventors tested whether fas-1 domain of βig-h3, which had been confirmed to have endothelial cell adhesion activity, is also involved in the adhesion and migration of endothelial cells (see Example 4). The results showed that the migration and adhesion of endothelial cells were inhibited by fas-1 domain in a dose-dependent manner (see FIGS. 4a and 4b). Particularly, it was shown that the fas-1 domain inhibited the adhesion and migration of endothelial cells at a much lower concentration than that of an YH18 synthetic peptide consisting of 18 amino acids including an YH motif. This confirms that the inhibitory effect of the fas-1 domain against the adhesion and migration of endothelial cells is significantly higher than that of the YH motif in view of their effective dose. It is considered that this superior inhibitory effect of fas-1 domain against the adhesion and migration of endothelial cells is because fas-1 domain forms a complete three-dimensional structure, whereas the YH18 synthetic peptide consisting of 18 amino acids including the YH motif cannot form the complete three-dimensional structure.

Meanwhile, an RGD motif acts as a recognition site for αvβ3 integrin. Thus, the present inventors tested whether fas-1 domain also inhibits the adhesion and migration of endothelial cells to other cellular matrix proteins containing the RGD motif as a ligand, in addition to βig-h3 (see FIG. 5). The results showed that fas-1 domain completely inhibited the adhesion and migration of endothelial cells to the RGD motif-containing proteins in a dose-dependent manner, whereas it only partially inhibits the adhesion and migration of endothelial cells to proteins containing no RGD motif as a ligand (see FIGS. 5a and 5b).

Furthermore, in the present invention, whether fas-1 domain inhibits the proliferation of endothelial cells was examined (see Example 6). The results showed that the proliferation of endothelial cells was inhibited by fas-1 domain in a dose-dependent manner (see FIG. 6). Moreover, the present inventors tested whether fas-1 domain are also involved in the apoptosis of endothelial cells (see Example 7), and the results showed that fas-1 domain specifically induced the apoptosis of endothelial cells by inducing caspase-3 activity (see FIGS. 7a and 7b). Also, it was shown that a αvβ3 integrin receptor was involved in the induction of endothelial cell apoptosis by fas-1 domain (see FIG. 8).

Afterward, the present inventors tested whether fas-1 domain inhibits angiogenesis (see Example 9). The results showed that angiogenesis was effectively by fas-1 domain both in vitro and in vivo (see FIGS. 9a and 9b). Also, it was shown that fas-1 domain inhibits angiogenesis at a much lower concentration than that of the YH18 synthetic peptide consisting of 18 amino acids including the YH motif.

Angiogenesis is an essential stage in the growth and metastasis of cancer cells (Weidner, N. et al., N. Engl. J. Med., 324:1-8, 1991). Thus, the present inventors tested whether fas-1 domain show an anticancer effect (see Example 10). The results showed that, although fas-1 domain did not affect the proliferation of cancer cells themselves, it inhibited the growth of tumors derived from the cancer cells. Also, it was shown that the tumor growth inhibitory effect of fas-1 domain was attributable to its anti-angiogenic effect (see FIGS. 10a to 10d).

As described above, in the present invention, it was found that the βig-h3 protein mediates the adhesion of endothelial cells by interaction with αvβ3 integrin, and its fas-1 domains inhibit the adhesion, migration and proliferation of endothelial cells and induce the apoptosis of endothelial cells. Also, it was found that the fas-1 domains inhibit angiogenesis and tumor growth. Such effects of the fas-1 domains are significantly superior to those of an YH motif conserved in the fas-1 domains.

To determine if fas-1 domains derived from other proteins in addition to the fas-1 domains of βig-h3 show an angiogenesis inhibitory effect, the present inventor performed an endothelial tube formation assay on the fas-1 domains of a stabilin-II protein (see Example 11). The results showed that the fas-1 domains derived from the stabilin-II protein also completely inhibited endothelial tube formation (see FIG. 11). This suggests that fas-1 domains derived from other proteins besides βig-h3 also show an angiogenesis inhibitory effect.

The present inventors tested a mechanism related to the proliferation and migration of endothelial cells by fas-1 domain (see Example 12). The results showed that fas-1 domain inhibited the proliferation and migration of endothelial cells by inhibiting a FAK-Raf-ERK/AKT signal transduction pathway (see FIG. 12).

The inventive polypeptide comprising the fas-1 domain has the following physiological activities:

First, the inventive polypeptide interacts with the αvβ3 integrin of endothelial cells.

Second, it inhibits the adhesion, migration and proliferation of endothelial cells.

Third, it induces the apoptosis of endothelial cells.

Fourth, it inhibits angiogenesis in vitro and in vivo.

Fifth, it inhibits the growth of tumors.

Accordingly, the present invention provides: a pharmaceutical composition for the inhibition of endothelial cell adhesion, migration and/or proliferation, which comprises as an active ingredient an isolated polypeptide comprising a fas-1 domain; a pharmaceutical composition for the induction of endothelial cell apoptosis, which comprises as an active ingredient an isolated polypeptide comprising a fas-1 domain; a pharmaceutical composition for the inhibition of angiogenesis, which comprises as an active ingredient an isolated polypeptide comprising a fas-1 domain; and a pharmaceutical composition for the treatment or prevention of angiogenesis-related diseases, which comprises as an active ingredient an isolated polypeptide comprising a fas-1 domain.

The angiogenesis-related diseases that can be treated or prevented according to the present invention include various cancers (tumors); vascular diseases such as hemangioma, angiofibroma, vascular malformation, arteriosclerosis, vascular adhesions, and edematous sclerosis; ocular diseases such as corneal graft neovascularization, neovascular glaucoma, diabetic retinopathy, angiogenic corneal disease, macular degeneration, pterygium, retinal degeneration, retrolental fibroplasia and granular conjunctivitis; inflammatory diseases such as rheumatoid arthritis, systemic Lupus erythematosus and thyroiditis; and dermatological diseases, such as psoriasis, capillarectasia, pyogenic granuloma, seborrheic dermatitis and acne (U.S. Pat. No. 5,994,292; Korean Patent Application Laid-Open No. 2001-66967; D'Amato R. J. et al., Ophtahlmol., 102:1261-1262, 1995; Arbiser J. L. J. Am. Acad. Derm., 34(3):486-497, 1996; O'Brien K. D. et al., Circulation, 93(4):672-682, 1996; Hanahan D. et al., Cell, 86:353-364, 1996). More preferred examples include cancers, arthritis, psoriasis, diabetic eye diseases, arteriosclerosis, and inflammation.

The pharmaceutical composition comprising the inventive polypeptide as an active ingredient may further comprise a pharmaceutically acceptable carrier, for example, a carrier for oral or parenteral administration. Examples of the carrier for oral administration include lactose, starch, cellulose derivatives, magnesium stearate, and stearic acid. For oral administration, the inventive polypeptide may be mixed with an excipient and used in various forms, including enteric tablets, buccal tablets, troches, capsules, elixirs, suspensions, syrups and wafers. Also, examples of the carrier for parenteral administration includes water, suitable oils, saline solution, aqueous glucose and glycol, and the inventive composition may further comprise stabilizers and conservatives. Suitable examples of the stabilizers include antioxidants, such as sodium hydrogensulfite, sodium bisulfite and ascorbic acid. Suitable examples of the preservatives include benzalkonium chloride, methyl- or propyl-paraben, and chlorobutanol. For other pharmaceutically acceptable carriers, reference may be made to the following literature: Remington's Pharmaceutical Sciences, 19th ed., Mack Publishing Company, Easton, Pa., 1995.

The pharmaceutical composition according to the present invention may be formulated in various forms for oral or parenteral administration. The formulations for parenteral administration are typically injection formulations, and preferably isotonic aqueous solution or suspension. The injection formulations may be prepared using suitable dispersing or wetting agents, and suspending agents, according to any technique known in the art. For example, the components may be formulated for injection by dissolving them in a saline or buffer solution. Examples of the formulations for oral administration include tablets and capsules, and these formulations may comprise diluents (e.g., lactose, dextrose, sucrose, mannitol, sorbitol, cellulose, and/or glycin) and lubricants (e.g., silica, talc, stearic acid and magnesium or calcium salts thereof, and/or polyethylene glycol), in addition to the active ingredient. The tablets may comprise binders such as magnesium aluminum silicate, starch paste, gelatin, tragacanth, methyl cellulose, sodium carboxymethylcellulose and/or polyvinylpyrrolidone, and occasionally, it may further comprise disintegrants such as starch, agar, alginate or a sodium salt thereof, absorbing agents, coloring agents, flavoring agents and/or sweetening agents on a case by case basis. These formulations may be prepared by a conventional method such as mixing, granulation or coating.

The pharmaceutical composition according to the present invention may additionally comprise aids such as preservatives, wettable powders, emulsifiers, salts for the regulation of osmotic pressure, and/or buffers, and other therapeutically useful materials. The pharmaceutical composition may be formulated according to a conventional method.

The total amount of the inventive polypeptide as an active ingredient in the inventive pharmaceutical composition can be administered to a subject as a single dose over a relatively short period of time, or can be administered using a fractionated treatment protocol where multiple doses are administered over a prolonged period of time. Although the content of the inventive polypeptide in the inventive pharmaceutical composition can vary depending on the severity of diseases, the inventive polypeptide can be generally administered several times a day at a dose of 10 μg-10 mg. However, the dose of the inventive polypeptide depends on many factors, including the age, weight, general health, sex, disease severity, diet and excretion of a subject, as well as the route of administration and the number of treatments to be administered. In view of these factors, any person skilled in the art would adjust the particular dose so as to obtain an effective dose for inhibiting angiogenesis, or for treating or preventing angiogenesis-related diseases. The pharmaceutical composition according to the present invention is not specifically limited in its formulation, administration route and administration mode insofar as it shows the effects of the present invention.

### BRIEF DESCRIPTION OF THE DRAWINGS

FIG. 1 shows the comparison between amino acid sequences containing an YH motif that is highly conserved in fas-1 domains derived from various proteins.

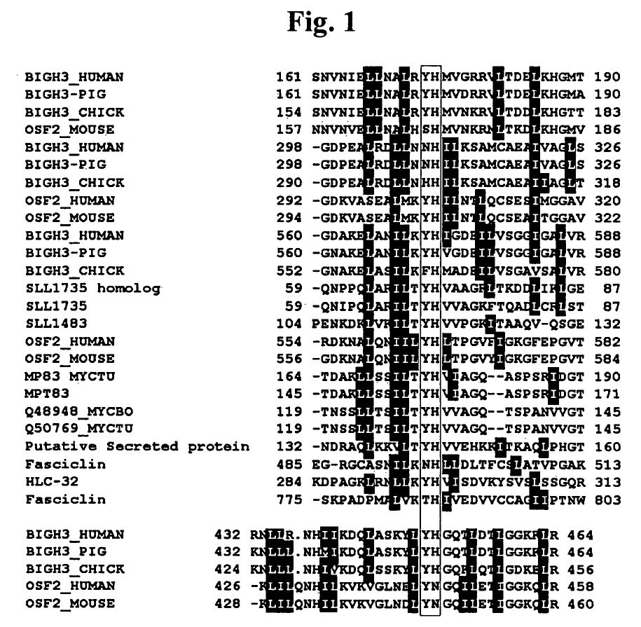

FIG. 2 is a schematic diagram (A) showing recombinant proteins containing each fas-1 domain of βig-h3, and a graphic diagram (B) showing the adhesion of HUVECs to the plate coated with βig-h3 or each of its fas-1 domains, in terms of absorbance.

BSA: bovine serum albumin as control

Wild-type: recombinant βig-h3 His-1-b protein containing all of four fas-1 domains

D-I: first fas-1 domain

D-II: second fas-1 domain

D-III: third fas-1 domain

D-IV: fourth fas-1 domain

FIG. 3a is a graphic diagram showing the inhibition of HUVECs adhesion to the plate coated with βig-h3 by function-blocking antibodies against various integrins.

BSA: plate coated with BSA

None: no treatment

α3: treated with P1B5 (antibody to α3)

α5: treated with P1D6 (antibody to α5)

αv: treated with P3G8 (antibody to αv)

β1: treated with 6S6 (antibody to β1)

β3: treated with B3A (antibody to β3)

αvβ3: treated with LM609 (antibody to αvβ3)

αvβ5: treated with P1F6 (antibody to αvβ5)

FIG. 3b is a graphic diagram showing the inhibition of HUVECs adhesion to the plate coated with each fas-1 domain by a function-blocking antibody against αvβ3 or αvβ5 integrin.

BSA: plate coated with BSA

D-I: first fas-1 domain of βig-h3

D-II: second fas-1 domain of βig-h3

D-III: third fas-1 domain of βig-h3

D-IV: fourth fas-1 domain of βig-h3

FIG. 3c shows the results of FACS analysis for the expression of integrin on the HUVECs surface using a function-blocking antibody against αvβ3 or αvβ5 integrin.

FIG. 3d shows the results of dose-dependent Western-immunoblotting analysis for the binding ability of biotin-βig-h3 to a HUVECs cell membrane (A), and for the inhibition of biotin βig-h3 binding to the HUVECs cell membrane by a function-blocking antibody against αvβ3 or αvβ5 integrin (B), in which the Western-immunoblotting analysis is conducted to identify a receptor for βig-h3 that is involved in endothelial cell adhesion. β-tubulin in FIG. 3d is an internal control for equal protein loading.

FIG. 4a is a graphic diagram showing the inhibition of endothelial cell adhesion according to the concentration of the fas-1 domain.

FIG. 4b is a graphic diagram showing the inhibition of endothelial cell migration according to the concentration of the fas-1 domain.

FIG. 5a is a graphic diagram showing the test results for the inhibitory effect of the fas-1 domain against the adhesion of endothelial cells to vitronectin, fibronectin or collagen, at various concentrations of the fas-1 domain.

FIG. 5b is a graphic diagram showing the test results for the inhibitory effect of the fas-1 domain against the migration of endothelial cells toward vitronectin, fibronectin or collagen, at various concentrations of the fas-1 domain.

FIG. 6 is a graphic diagram showing the inhibition of endothelial cell proliferation according to the concentration of the fas-1 domain.

FIG. 7a shows the results of FACS analysis using annexin V staining, for the apoptosis of endothelial cells (HUVECs) or melanoma cells (B16F10) by the fas-1 domain.

FIG. 7b is a graphic diagram showing caspase-3 activity that is induced by the fas-1 domain in a dose-dependent manner.

FIG. 8 shows the results of FACS analysis using annexin V staining, which was conducted to identify integrins mediating the induction of endothelial cell apoptosis by the fas-1 domain.

pc/HEK293: HEK293 cells transfected with a pcDNA3 vector

β3/HEK293: HEK293 cells transfected with a β3 integrin expression vector

β5/HEK293: HEK293 cells transfected with a β5 integrin expression vector

FIG. 9a is a photograph (A) and a graph (B), which show that HUVECs tube formation is inhibited by the fas-1 domain.

BSA: treated with BSA as a control to the fas-1 domain

None: no treatment

FIG. 9b is a photograph (A) showing the inhibition of angiogenesis by the fas-1 domain, a photograph (B) showing a section stained with H&E, and a graphic diagram (C) showing the result of measurement for the number of blood vessels.

FIG. 10a is a graphic diagram showing the effect of fas-1 against the proliferation of fas-1-overexpressing melanoma cells (fas-1/B16F10).

FIG. 10b is a graphic diagram showing the effect of fas-1 against the growth of tumors derived from fas-1-overexpressing melanoma cells (fas-1/B16F10).

FIG. 10c is a graphic diagram showing the effect of fas-1 against the growth of melanoma.

FIG. 10d is a photograph (A) and a graphic diagram (B), which show the result of measurement for the number of blood vessels in melanoma treated with fas-1 domain by immuno-staining.

FIG. 11 is a photograph (A) and a graphic diagram (B), which show that HUVECs tube formation is inhibited by various fas-1 domains.

BSA: control to βig-h3 D-II

D-II: second fas-1 domain of βig-h3

Nus: control to Nus-fas-3 and Nus-fas-7

Nus-fas-3: third fas-1 domain of stabilin-II

Nus-fas-3: seventh fas-1 domain of stabilin-II

FIG. 12 is a Western blot photograph showing test results for the phosphorylation of enzymes involved in a FAK-Raf-ERK/AKT signal transduction pathway after treatment with the fas-1 domain.

### BEST MODE FOR CARRYING OUT THE INVENTION

Hereinafter, the present invention will be described in detail by examples. It will however be obvious to a person skilled in the art that the present invention is not limited to or by the examples.

### Example 1

### Expression and Purification of βig-h3 Protein and its fas-1 Domains

1-1: Construction of Expression Vector

Previously, the present inventors reported recombinant proteins comprising βig-h3 and each of its fas-1 domains (Kim, J.-E. et al., J. Biol. Chem., 275:30907-30915, 2000; and Korean Patent Registration No. 10-0382042). Thus, βig-h3 and its fas-1 domains were prepared in the same manner as described in the report.

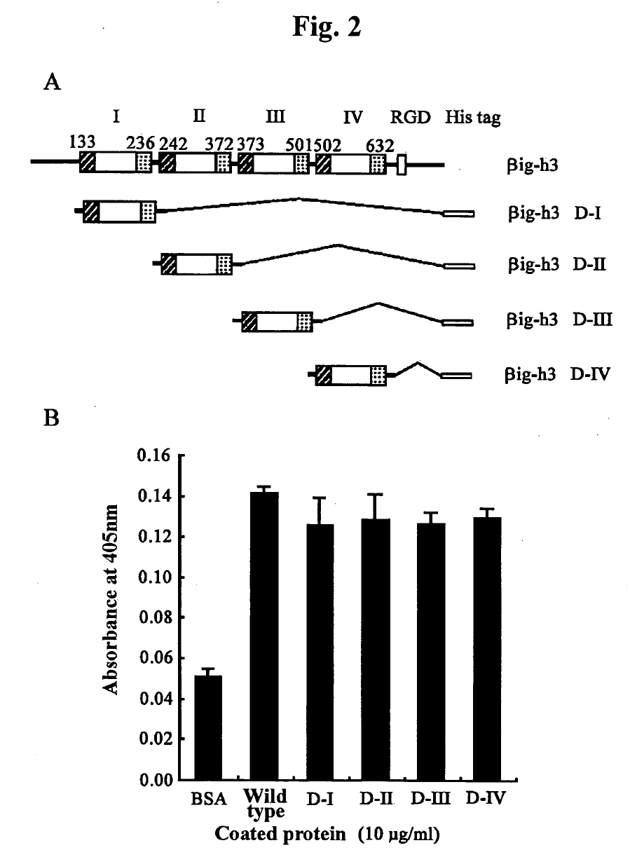

Concretely, a recombinant human βig-h3 protein (hereinafter, referred to as “βig-h3 His-1-b”) that expresses all of four fas-1 domains was prepared using a pHis-β-b vector. The pHis-β-b vector had been prepared by inserting an Asp718-BglII fragment (obtained by partially deleting the amino terminal region of βig-h3 cDNA) into the EcoRV/EcoRI sites of pET-29β. Also, to express recombinant proteins containing each fas-1 domain of human βig-h3, the present inventors PCR-amplified four cDNA fragments of βig-h3, which include the first fas-1 domain (βig-h3 D-I), the second fas-1 domain (βig-h3 DII), the third fas-1 domain (βig-h3 D-III) or the fourth fas-1 domain (βig-h3 D-IV), respectively (see A of FIG. 2).

Then, each of the PCR products was cloned into the EcoRV/XhoI sites of a pET-29b(+) vector (Novagen; Madison, Wis.). The constructed expression vectors were named “pβig-h3 D-I”, “pβig-h3 D-II”, “pβig-h3 D-III” and “pβig-h3 D-IV”, respectively. The amino acid sequences of the four fas-1 domains of βig-h3 are as set forth in SEQ ID NO: 61 to SEQ ID NO: 64, respectively.

1-2: Transformation of E. coli and Purification of Recombinant Protein

E. coli BL21 DE3 cells were transformed with each of the expression vectors constructed in Example 1-1. The transformed E. coli cells were incubated in LB medium containing 50 μg/ml kanamycin. To induce the expression of each recombinant protein, when the absorbance of the culture reached 0.5-0.6 at a 595 nm, the culture was added with 1 mM IPTG (isopropyl-β-D-(−)thiogalactopyranoside) and further incubated at 37° C. for three hours. Next, purification of the expressed proteins was conducted according to the method described by Kim, J.-E. et al., J. Cell. Biochem., 77:169-187, 2000. For this purpose, the cells were collected by centrifugation and resuspended in buffer (50 mM Tris-HCl (pH 8.0), 100 mM NaCl, 1 mM EDTA, 1% Triton X-100, 1 mM PMSF, 0.5 mM DTT). The cell suspension was disrupted by sonication. The proteins expressed in the form of an inclusion body were dissolved in an 8M urea-denaturation buffer, and the denaturated proteins were purified with a Ni-NTA resin (Qiagen). The recombinant proteins were eluted in 200 mM imidazole solution, and purified by dialysis against 20 mM Tris-HCl buffer containing 50 mM sodium chloride in a stepwise manner from high to low urea concentration. The expressed and purified proteins were analyzed by SDS-PAGE (data not shown). Unlike the recombinant protein βig-h3 His-1-b containing all of the four fas-1 domains, only two recombinant proteins containing the second- or fourth fas-1 domain were synthesized in a water-soluble form and thus did not require a modification step. Also, they could be easily obtained in large amounts.

Meanwhile, the E. coli transformed with the expression vectors pHis-β-b, pβig-h3 D-II and pβig-h3 DIV were termed “E. coli BL21/His-β-b”, “E. coli BL21/Hisβ-g” and “E. Coli BL21/Hisβ-e”, respectively, and deposited in the Korean Collection for Type Cultures (KCTC), Korean Research Institute of Bioscience and Biotechnology, under accession numbers KCTC 18008P, KCTC 18010P and KCTC 18009P, respectively, on Apr. 25, 2000.

### Example 2

### Test of Endothelial Cell Adhesion Activity of βig-h3 and its fas-1 Domains

Cell adhesion assay was performed according to the method described by Kim, J.-E. et al., J. Biol. Chem., 277:46159-46165, 2002. For this purpose, 10 μg/ml of each of the recombinant proteins (βig-h3 His-1-b, βig-h3 D-I, βig-h3 D-II, βig-h3 D-III and βig-h3 D-IV) prepared in Example 1 was placed into a flat-bottomed 96-well ELISA (enzyme-linked immunosorbent assay) plate (Costar, Corning Inc., NY) and then incubated overnight at 4° C., to coat the surface of the plate. As a control, 2% BSA was coated on the plate. Then, the plate was treated with PBS (phosphate-buffered saline) containing 2% BSA, and blocked at room temperature for one hour.

Meanwhile, HUVECs (human umbilical vein endothelial cells; Clonetics, San Diego, Calif.) were incubated in EGM medium (Clonetics, San Diego, Calif.) containing 2% FBS (Fetal Bovine Serum) under a condition of 37° C. and 5% CO₂. The incubated cells were suspended in medium at a density of 3×10⁵ cells/ml, and 0.1 ml of the cell suspension was added to each well of the plate. Next, the cells were incubated at 37° C. for 30 minutes and washed one time with PBS buffer to remove cells which had not been attached to the plate. Attached cells were added with 50 mM citrate buffer (pH 5.0) containing 3.75 mM p-nitrophenyl-N-acetyl β-D-glycosaminide and 0.25% Triton X-100, and incubated at 37° C. for one hour. Thereafter, 50 mM glycine buffer (pH 10.4) containing 5 mM EDTA was added to block the activity of the enzyme. The absorbance was measured at a 405-nm in a Bio-Rad model 550 microplate reader. Here, the higher the number of cells adhered to the plate, the higher the absorbance.

The results showed that, as shown in B of FIG. 2, βig-h3 mediated the adhesion of endothelial cells, and each of the fas-1 domains of βig-h3 also mediated the adhesion of endothelial cells with an almost equal activity to that of βig-h3.

### Example 3

### Identification of Integrins that are Involved in Adhesion of Endothelial Cells to βig-h3

3-1: Test 1 for Identification of Integrin Receptors

In order to identify integrins that are involved in the adhesion of endothelial cells to βig-h3, the present inventors have conducted a cell adhesion inhibition assay using various antibodies that specifically blocks the function of integrins.

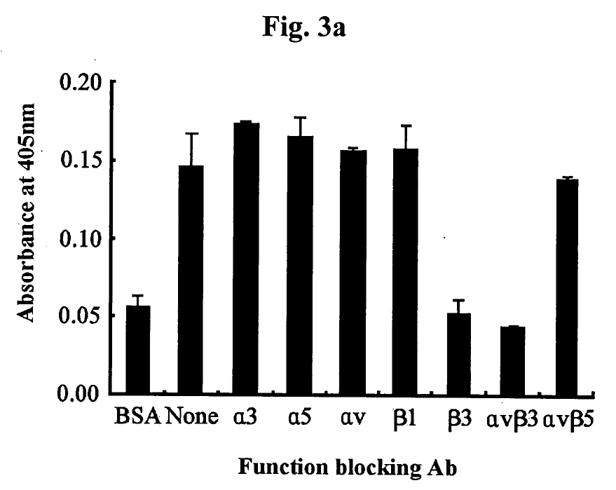

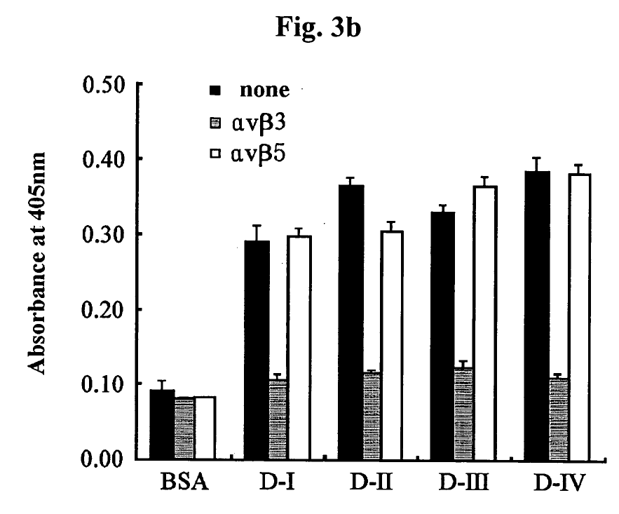

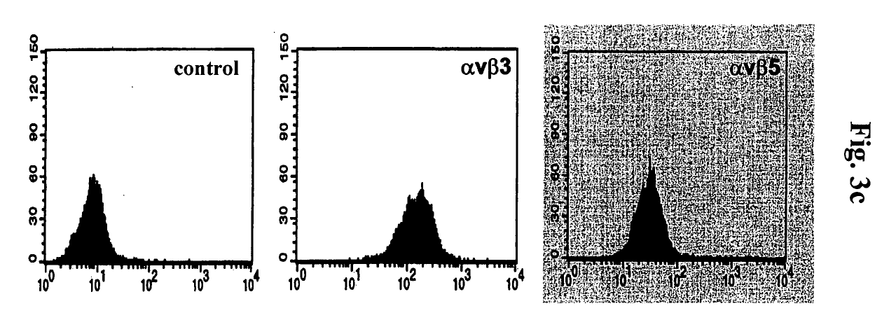

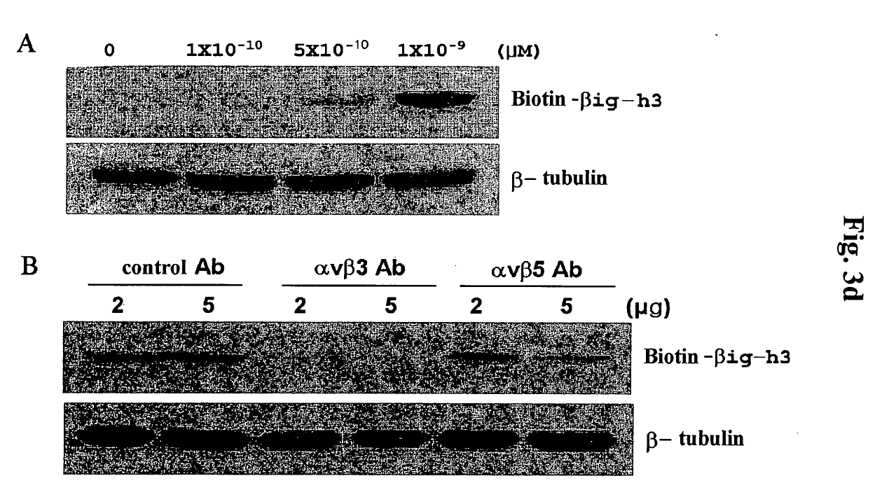

For this purpose, 5 μg/ml of monoclonal antibodies specific to different types of integrin (Chemicon, International Inc, Temecula, Calif.) was preincubated at 37° C. for 30 minutes with HUVECs in 0.1 ml of the cell suspension (3×10⁵ cells/ml). The following antibodies were used in this test: P1B5 (antibody to α3), P1D6 (antibody to α5), P3G8 (antibody to αv), 6S6 (antibody to β1), B3A (antibody to β3), LM609 (antibody to αvβ3) and P1F6 (antibody to αvβ5). A culture which had not been preincubated with the antibody was used as a control. Then, the incubated cells were transferred onto plates precoated with the recombinant protein βig-h3 His-1-b and incubated at 37° C. for 30 minutes. The attached cells were then quantified in the same manner as in Example 2. The results showed that, as shown in FIG. 3a, the adhesion of endothelial cells to βig-h3 was inhibited specifically by the antibodies to αvβ3 integrin and β3 integrin, but it was not inhibited by the antibodies to other integrins, including α3 and α5.

3-2: Test 2 for Identification of Integrin Receptors

In Example 2 above, it was confirmed that βig-h3 and also its fas-1 domains mediate the adhesion of endothelial cells. Thus, in order to identify an integrin receptor for each of the fas-1 domains of βig-h3, the present inventors coated a plate surface with each of the fas-1 domains of βig-h3 and conducted a cell adhesion inhibition assay.

The results showed that, as shown in FIG. 3b, the adhesion of endothelial cells to each of the fas-1 domains was inhibited specifically by the antibody to αvβ3, but it is not inhibited by the antibody to αvβ5.

3-3: Confirmation of Integrins that are Expressed on Surface of Endothelial Cells

To confirm that HUVECs express both αvβ3 and αvβ5 integrin on their surface, the present inventors conducted an FACS analysis using monoclonal antibodies specific to the two integrins.

For this purpose, a plate in which HUVECs had been grown to confluence was treated with PBS buffer containing 0.25% trypsin and 0.05% EDTA to detach the cells from the plate surface. The cells were washed two times with PBS buffer and resuspended in PBS buffer. The cell suspension was added with an anti-αvβ3 integrin antibody (LM609; Chemicon, International Inc, Temecula, Calif.) or an anti-αvβ5 integrin antibody (PIF6; Chemicon, International Inc, Temecula, Calif.) and incubated at 4° C. for one hour. The cells were then further incubated for one hour at 4° C. with 10 μg/ml of a FITC-conjugated secondary goat antimouse IgG antibody (Santa Cruz Biotechnology, Inc., CA). The resulting cells were analyzed at 488 nm on the flow cytometer FACSCalibur system (Becton Dickinson, San Jose, Calif.) equipped with a 5-watt laser. A control was incubated with a secondary antibody alone.

The results showed that, as shown in FIG. 3c, HUVECs expressed both the αvβ3 integrin and the αvβ5 integrin. However, the expression level of the αvβ5 integrin was far less than that of the αvβ3 integrin.

3-4: Test 3 for Identification of Integrin Receptors

To confirm that HUVECs adhesion that is mediated by βig-h3 depends on αvβ3 integrin, the present inventors tested the binding affinity of βig-h3 in the presence of an antibody that specifically blocks the function of the integrin. This binding assay was performed according to the method described by Maile, L. A., et al., J. Biol. Chem., 275:23745-23750, 2002.

First, HUVECs were suspended in medium at a density of 1×10⁵ cells/ml. 1 ml of the cell suspension was preincubated with anti-αvβ3 antibody (LM609) or anti-αvβ5 antibody (P1F6) at 37° C. for 30 minutes. Thereafter, the preincubated cells were incubated with biotinylated βig-h3 (hereinafter, referred to as “biotin-βig-h3”) in a serum-free medium containing 0.1% BSA at 4° C. for 5 hours. The biotin-βig-h3 was added at concentrations of 1×10⁻¹⁰, 1×10⁻⁹ and 5×10⁻⁹ μM, respectively. Then, the cells were washed three times with PBS buffer (pH 7.4), and dissolved at 4° C. in ice-cold buffer A (10 mM Tris-Cl, pH 7.4, 150 mM NaCl, 1% Nonidet P-40, 1% sodium deoxycholate, 0.5% SDS, 0.02% sodium azide, 1 mM EDTA, 1 mM phenylmethylsulfonyl fluoride). The cell lysates were clarified by centrifugation at 13,000 rpm for 30 minutes at 4° C. Equal amounts of protein were then separated by SDS-PAGE, 8% gel. The amount of biotin-βig-h3 was determined by Western immuno-blotting.

To visualize the biotin-βig-h3, the membranes were incubated with HRP (hoseradish peroxidase; Amersham Biosciences)-conjugated streptavidin. Next, Binding of the peroxidase-labeled antibody was visualized using ELC (enhanced chemiluminesence; Amersham Biosciences). As an internal control, a β-tubulin protein was subjected to Western blotting to verify equal protein loading.

The results showed that βig-h3 was bound to HUVECs surface in a dose-dependent manner (see A of FIG. 3d), and its binding was specifically inhibited only by the antibody to αvβ3 integrin (see B of FIG. 3d).

Such results suggest that each of the fas-1 domains of βig-h3 contains a motif that mediates the adhesion of endothelial cells through the “αvβ3 integrin” other than the αvβ5 integrin.

### Example 4

### Assay of Inhibition of Endothelial Cell Adhesion and Migration by fas-1 Domains

4-1: Cell Adhesion Assay

The present inventors tested whether the fas-1 domains inhibit the adhesion of endothelial cells, which is mediated by βig-h3 (Nam, J. O. et al., J. Biol. Chem., 278:25902-25909, 2003).

HUVECs were suspended in medium at a density of 3×10⁵ cells/ml, and 0.1 ml of the cell suspension was added with the fourth fas-1 domain of βig-h3 (βig-h3 D-IV) prepared in Example 1, at concentrations of 5 μM, 10 μM and 20 μM, respectively, and preincubated at 37° C. for 30 minutes. The preincubated cells were added to each well of the 96-well plate precoated with a recombinant βig-h3 His-1-b protein. Then, the cells were tested in the same manner as in Example 2. A control was treated with 2% BSA.

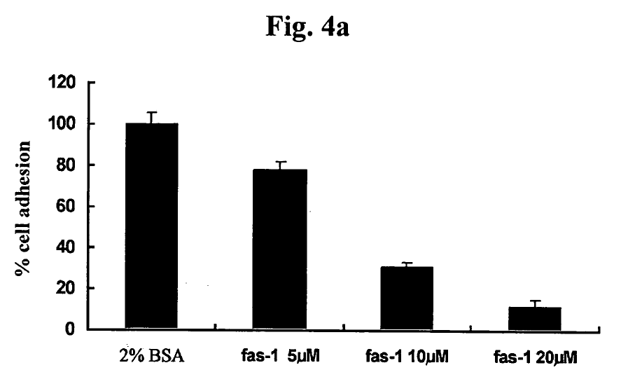

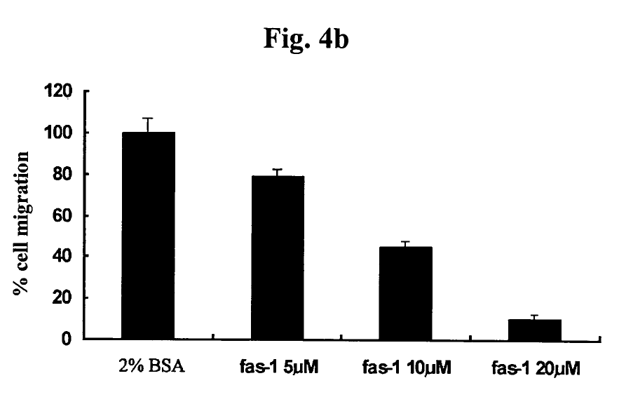

The results showed that, as shown in FIG. 4a, the adhesion of endothelial cells, which is mediated by βig-h3, was inhibited even in the case of treatment with 5 μM of the fas-1 domain, and was inhibited in a dose-dependent manner. Furthermore, an inhibitory effect of endothelial cell adhesion in the case of treatment with 20 μM of the fas-1 domain was higher than the inhibitory effect in the case of treatment with 1,000 μM of a YH motif-containing peptide fragment of 18 amino acids present in the fas-1 domain, in which the inhibitory effect of the peptide fragment had been confirmed previously by the present inventors (Nam, J. O. et al., J. Biol. Chem., 278:25902-25909, 2003).

4-2: Cell Migration Assay

The present inventors tested whether the fas-1 domain has the activity to inhibit the migration of endothelial cells (Nam, J. O. et al., J. Biol. Chem., 278:25902-25909, 2003). Cell migration assay was performed in a transwell plate (8 μm pore size, Costar, Cambridge, Mass.). The undersurface of the membrane was coated with the recombinant βig-h3 His-1-b (10 μg/ml) protein prepared in Example 1, at 4° C., and then, blocked for 1 hour at room temperature with PBS buffer containing 2% BSA. Meanwhile, HUVECs were added with the fas-1 domain (βig-h3 D-IV) prepared in Example 1, and preincubated at 37° C. for 30 minutes. A control was added with 2% BSA. Then, the HUVECs preincubated with the fas-1 domain were suspended in medium at a density of 3×10⁵ cells/ml, and 0.1 ml of the cell suspension was added to the upper compartment of the filter. The cells were allowed to migrate at 37° C. for 6-8 hours.

Migration was terminated by removing the cells from the upper compartment of the filter with a cotton swab. The filters were fixed with 8% glutaraldehyde and stained with crystal violet. The extent of cell migration was determined by light microscope, and within each well, cell counting was done in nine randomly selected fields HPF (Microscopic high power fields, x200).

The results showed that, as shown in FIG. 4b, the migration of endothelial cells, which is promoted by βig-h3, was inhibited by the fas-1 domain in a dose-dependent manner. Furthermore, an inhibitory effect of endothelial cell migration in the case of treatment with 20 μM of the fas-1 domain was higher than the inhibitory effect in the case of treatment with 1,000 μM of a YH motif-containing peptide fragment of 18 amino acids present in the fas-1 domain, in which the inhibitory effect of the peptide fragment had been confirmed previously by the present inventors (Nam, J. O. et al., J. Biol. Chem., 278:25902-25909, 2003).

The above results confirm that the fas-1 domain is a more powerful inhibitory effect against the adhesion and migration of endothelial cells, as compared to the YH motif-containing peptide fragment of 18 amino acids present in the fas-1 domain. This is because the fas-1 domain has a complete three-dimensional structure while the YH motif does not form the complete three-dimensional structure, so that the fas-1 domain has a higher inhibitory effect than that of the YH motif.

### Example 5

### Inhibitory Effect of fas-1 Domain Against Adhesion and Migration of Endothelial Cells to Other Cellular Matrix Proteins

In Example 4 above, the present inventors confirmed that the fas-1 domain significantly inhibited the adhesion and migration of endothelial cells to βig-h3. The carboxyl terminal region of the βig-h3 protein contains an RGD motif that acts as a ligand recognition site for αvβ3 integrin. Thus, the present inventors tested whether the fas-1 domain can also inhibit the adhesion and migration of endothelial cells to other cellular matrix proteins containing the RGD motif as a ligand, in addition to the βig-h3 protein. The test was performed using fibronectin, vitronectin and collagen (control), which play an important role in the formation of new blood vessels by mediating the adhesion and migration of endothelial cells.

5-1: Cell Adhesion Assay

Flat-bottomed 96-well ELISA (enzyme-linked immunosorbent assay) plates (Costar, Corning Inc., NY) were incubated overnight at 4° C. with 10 μg/ml of fibronectin (Promega Inc., USA), vitronectin (Promega Inc., USA) and collagen, respectively, to coat the well surface. Then, a test was performed in the same manner as in Example 4-1 above.

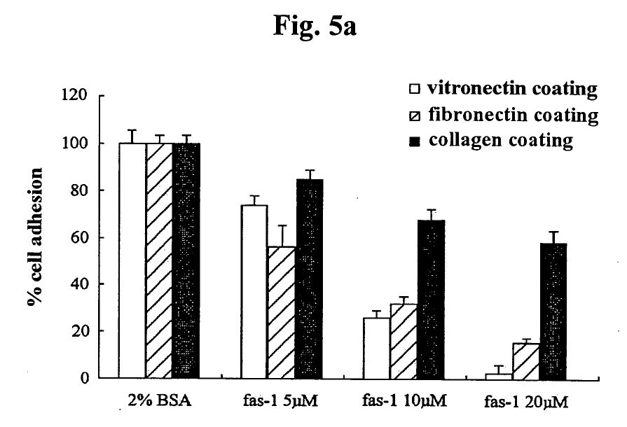

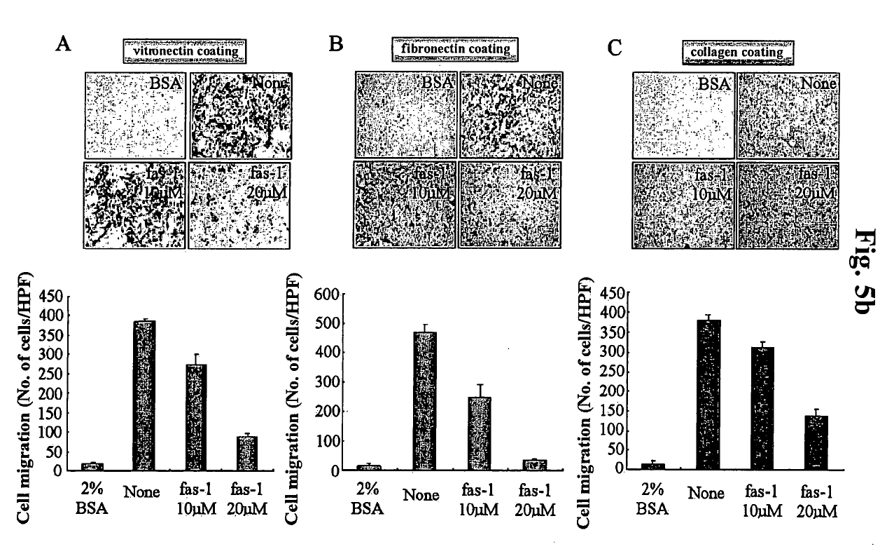

The results showed that, as shown in FIG. 5a, the adhesion of HUVECs to fibronection and vitronectin, which contain the RGD motif as a ligand, was completely inhibited by the fas-1 domain in a dose-dependent manner, whereas the adhesion of HUVECs cells to collagen containing no RGD motif as a ligand was only partially inhibited by the fas-1 domain.

5-2: Cell Migration Assay

The undersurface of a transwell plate was coated with each of fibronectin, vitronectin and collagen, and then, a test was performed in the same manner as in Example 4-2 above.

The results showed that, as shown in FIG. 5b, the migration of HUVECs toward fibronectin and vitronectin, which contain the RGD motif as a ligand, was completely inhibited by the fas-1 domain in a dose-dependent manner. However, it was shown that the migration of HUVECs to collagen containing no RGD motif as a motif was partially inhibited by the fas-1 domain.

The above results suggest that the fas-1 domain also has an inhibitory effect against the adhesion and migration of endothelial cells to other RGD-dependent integrins.

### Example 6

### Assay of Endothelial Cell Proliferation Inhibition by fas-1 Domain

To examine if the fas-1 domain is involved in the proliferation of endothelial cells, the present inventors added the fas-1 domain to endothelial cells, and then, tested whether the proliferation of the endothelial cells is inhibited.

HUVECs were suspended in medium at a density of 3×10⁴ cells/ml, and 0.1 ml of the cell suspension was added to each well of 96-well plate and incubated at 37° C. overnight. The cells were washed with PBS buffer, and then incubated at 37° C. for 24 hours in serum-free medium. Thereafter, 0.2% FBS-containing medium containing the indicated concentrations of the fas-1 domain (βig-h3 D-IV) prepared in Example 1 was added to each well of the plate and incubated at 37° C. for 48 hours. A control was added with 2% BSA instead of the fas-1 domain. 50 μl of 0.5 mg/ml thiazolyl blue (MTT; Sigma) solution was added to each well, and allowed to react at 37° C. for 4 hours. After the reaction, the medium was removed, and each well was added with DMSO and left to stand at room temperature for a few minutes. Thereafter, the absorbance was measured at 570 nm in a Bio-Rad model 550 microplate reader.

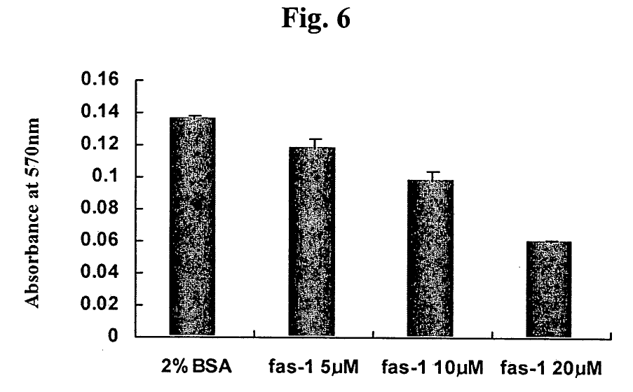

The results showed that, as shown in FIG. 6, the proliferation of endothelial cells was inhibited by fas-1 domain in a dose-dependent manner, whereas it was not inhibited by BSA.

### Example 7

### Assay of Induction of Endothelial Cell Apoptosis by fas-1 Domain

To examine whether the fas-1 domain is involved in endothelial cell apoptosis, the present inventors added the fas-1 domain to endothelial cells and then tested whether the apoptosis of the endothelial cells is induced by fas-1 domain.

7-1: Annexin V Staining

Annexin V is a protein binding to calcium-dependent phospholipid, and known to have a strong affinity for phosphatidylserine such that apoptosis can be assayed. For this reason, the present inventors performed a test using an annexin V staining kit (Santa Cruz Biotechnology Inc.).

Each of human umbilical vein endothelial cells (HUVECs) and melanoma cells (B16F10) was suspended in medium at a density of 5×10⁵ cells/ml, added to each well of 5-well plate and incubated at 37° C. overnight. On the next day, the cells were washed one time with PBS buffer, then added with serum-free medium and incubated at 37° C. for 24 hours. 0.2% FBS-containing fresh medium was treated with the fas-1 domain (βig-h3 D-IV) prepared in Example 1, at various concentrations, then added to each well of the plate and incubated at 37° C. for 48 hours. A negative control was added with PBS, a positive control was incubated with TNF-α or Vitamin C, which are known to induce the apoptosis of endothelial cells and melanoma cells. FITC (fluorescein isothiocyanate)-conjugated annexin V antibodies were added and allowed to react at 4° C. for one hour. After completion of the reaction, unbound annexin V antibodies were removed with PBS buffer, and the apoptosis of the cells was analyzed using FACS (BD Biosciences).

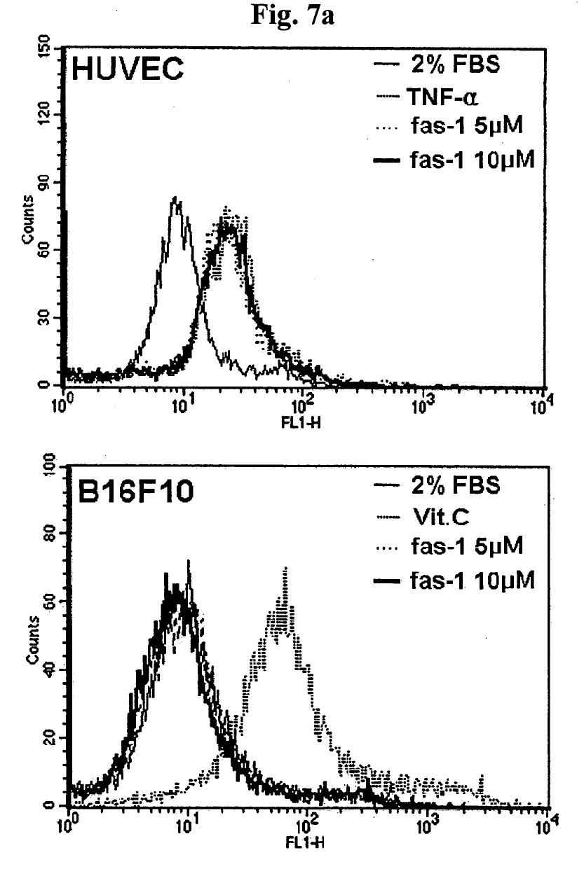

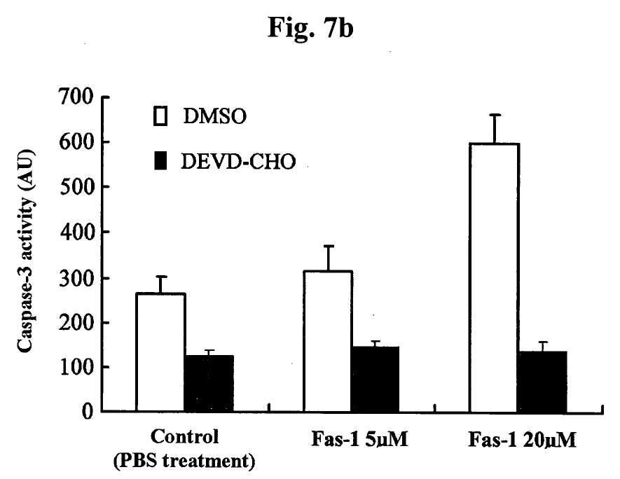

The results showed that, as shown in FIG. 7a, the fas-1 domain induced the apoptosis of HUVECs in a dose-dependent manner, whereas it did not induce the apoptosis of B16F10 cells. This suggests that the apoptosis of cells by the fas-1 domain is specific only for endothelial cells.

7-2: Measurement of Caspase-3 Activity

With respect to the induction of endothelial cell apoptosis (active cell death) by the fas-1 domain, which had been confirmed in Example 7-1 above, the present inventors measured the activity of caspase-3, which is activated when apoptosis occurs, in the cells treated with the fas-1 domain.

HUVECs were spread onto a 100-mm plate (10 ml culture solution) with M199 medium containing 10% FBS, followed by incubation in a 37° C. incubator for 12 hours. After the incubation, the medium was removed from the plate, the cells were added with fresh medium containing 0.1% FBS and further incubated for 24 hours. The cell-containing medium was treated with 5 μM or 20 μM of the fas-1 domain (βig-h3 D-IV) prepared in Example 1. A control was treated with PBS buffer. At 12 hours and 24 hours after the treatment, the cells were harvested. The caspase-3 activity of the cells was measured with an EnzCheck Caspase-3 Assay Kit(#2) (Molecular Probe Co., Eugene, Oreg., USA). Moreover, to verify that measured values are attributed to caspase-3 activity, each of the samples was treated with DEVD-CHO (Molecular Probe Co., Eugene, Oreg., USA) as a caspase-3-specific inhibitor. The control was treated with DMSO.

The results showed that, as shown in FIG. 7b, the caspase-3 activity of the control (treated with PBS) at 24 hours after the treatment was about 250 AU (random value). However, the caspase-3 activity of the group treated with the fas-1 domain were 300 AU at 5 μM, and 600 AU at 20 μM, indicating that the caspase-3 activity is increased in a dose-dependent manner. Also, it was shown that the caspase-3 activities of the test groups and the control group were inhibited by treatment with DEVD-CHO as a caspase-3-specific inhibitor. The above results suggest that the fas-1 domain induces the apoptosis of endothelial cells by increasing the activity of caspase-3.

### Example 8

### Identification of Receptors that are Involved in Induction of Endothelial Cell Apoptosis by fas-1 Domain

To identify an integrin that mediates the induction of endothelial cell apoptosis by the fas-1 domain, the present inventors performed a test using HEK293 cells (β33/HEK293 and β5/HEK293) which had been transfected such as to express αvβ3 integrin or αvβ5 integrin.

For this purpose, a β3/HEK293 cell that expresses αvβ3 integrin was prepared in the following manner. In order to construct a human β3 integrin expression vector, RT-PCR using human placenta poly(A)⁺ RNA as a template was first performed to produce 2.4-kb β3 cDNA (Chandrika, S. K. et al., J. Biol. Chem., 272:16390-16397, 1997). The amplified β3 cDNA was digested with HindIII/XbaI, and then cloned into a pcDNA3 vector (Invitrogen). The resulting vector was named “β3/pcDNA3”. Thereafter, 1 μg of the β3/pcDNA3 vector was introduced into HEK293 cells (ATCC, catalog No. CRL 1573) by lipofectamin (Gibco). As the vector contains a G418 selection marker, a stable transfected cell was screened using 1 mg/ml of G418. The cell transfected with the β3/pcDNA3 vector was named “3/HEK293”.

Meanwhile, a β5/HEK293 cell that expresses αvβ5 integrin was prepared in the following manner. In order to construct a human β5 integrin expression vector, RT-PCR using human placenta poly(A)⁺ RNA as a template was first performed to prepare a 2.8-kb β5 cDNA (Suzuki et al., Proc. Natl. Acad. Sci. USA, 87:5354-5358, 1990). The amplified β5 cDNA was digested with EcoRI, and then cloned into a pcDNA3 vector (Invitrogen). The resulting vector was named “β5/pcDNA3”. Thereafter, 1 μg of the β5/pcDNA3 vector was introduced into HEK293 cells (ATCC, catalog No. CRL 1573) by lipofectamin (Gibco). A stable transfected cell was screened using 1 mg/ml of G418. The cell transfected with the β5/pcDNA3 vector was named “β5/HEK293”.

A control cell was transfected with a pcDNA3 vector containing no β3 cDNA or β5 cDNA fragment, and named “pc/HEK293”.

pc/HEK293 cells, β3/HEK293 cells and β5/HEK293 cells were stained with annexin V to identify an integrin that mediates the induction of endothelial cell apoptosis by the fas-1 domain. Each of the cells was suspended in medium at a density of 5×10⁵ cells/ml, added to each well of 6-well plated and incubated at 37° C. overnight. Following this, a test was performed in the same manner as in Example 7-1 above.

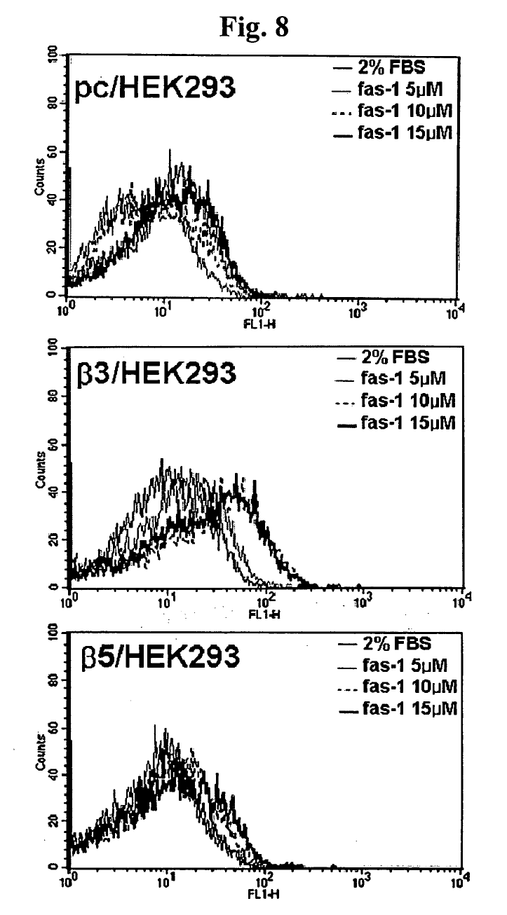

The results showed that, as shown in FIG. 8, the apoptosis of the β3/HEK293 cells was induced by the fas-1 domain in a dose-dependent manner, whereas the apoptosis of the pc/HEK293 cells and the β5/HEK293 cells was not induced by the fas-1 domain. Such results suggest that the fas-1 domain induces the apoptosis of endothelial cell through αvβ3 integrin.

### Example 9

### Assay of Angiogenesis Inhibition by fas-1 Domain

Through Examples 4, 6 and 7 above, it was confirmed that the fas-1 domain not only inhibits the adhesion, migration and proliferation of endothelial cells but also induces the apoptosis of endothelial cells. Thus, in the present invention, whether the fas-1 domain also inhibits angiogenesis was tested in vitro and in vivo (Nam, J. O. et al., J. Biol. Chem., 278:25902-25909, 2003).

9-1: Endothelial Tube Formation Assay

First, 100 μl of Matrigel (Chemicon, International Inc, Temecula, Calif.) was added to each well of a 96-well plate and allowed to polymerize. HUVECs were suspended in medium at a density of 3×10⁵ cells/ml, and 0.1 ml of the cell suspension was added to each well of the plate coated with Matrigel. At this time, 5 μM or 10 μM of the fas-1 domain (βig-h3 D-IV) prepared in Example 1 above was added together. A control was either treated with BSA or had no treatment. Thereafter, the cells were incubated at 37° C. for 16-18 hours. The cells were photographed, and endothelial tubes were counted and averaged.

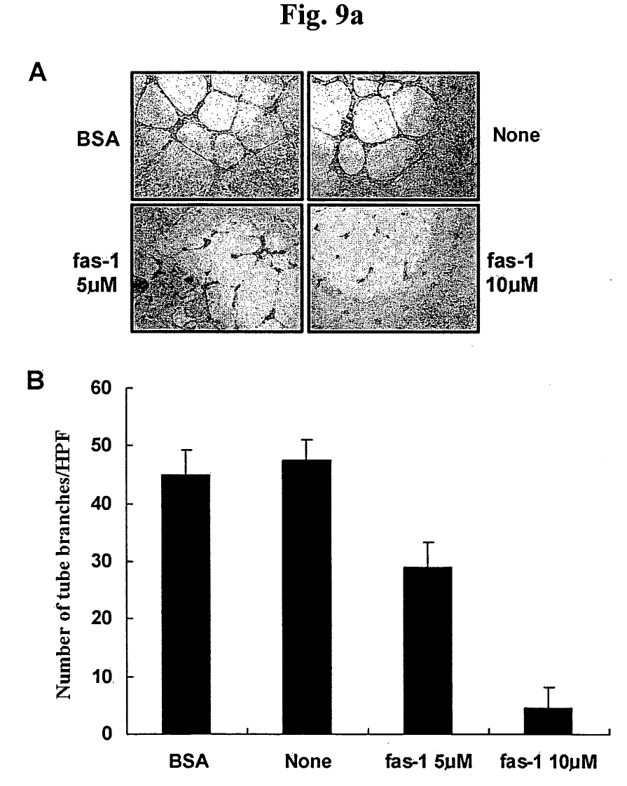

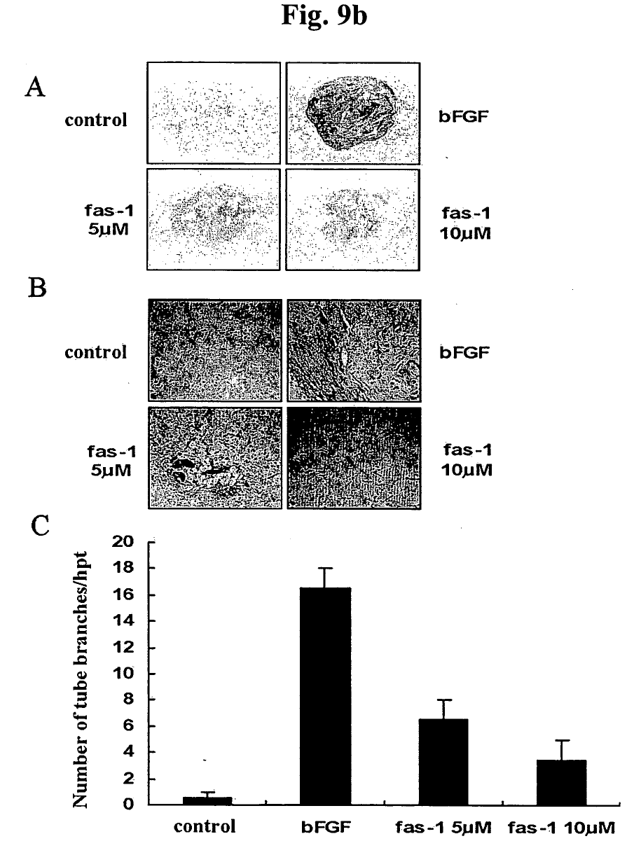

The results showed that, as shown in FIG. 9a, the fas-1 domain inhibited the formation of endothelial tubes on Matrigel in a dose-dependent manner. Also, an in vitro angiogenesis inhibitory effect in the case of treatment with 10 μM of the fas-domain was much higher than that in treatment with 1 mM of the YH motif-containing peptide fragment of 18 amino acids present in the fas-1 domain, in which the inhibitory effect of the peptide fragment had been confirmed previously by the present inventors (Nam, J. O. et al., J. Biol. Chem., 278:25902-25909, 2003).

9-2: Matrigel Plug Assay

An in vivo Matrigel plug assay was performed according to the method described by Maeshima, Y. et al., J. Biol. Chem., 275:23745-23750, 2000. 5-6 week old male C57/BL6 mice purchased from Hyochang scientific company, Korea, were used.

First, Matrigel (BD Biosciences, MA) was mixed with 20 units/ml heparin, 150 ng/ml bFGF (basic fibroblast growth factor, R&D system, International, Inc), and 5 μM or 10 μM of the fas-1 domain (βig-h3 D-IV). As a control was treated with PBS. The Matrigel mixture was injected subcutaneously to the C57/BL6 mice. After 7 days, the mice were sacrificed, and the Matrigel plugs were removed and fixed in 4% paraformaldehyde. The Matrigel plugs were embedded in paraffin, sectioned and stained with H&E. Their sections were examined by light microscope, the number of blood vessels from 4-6 HPF (high power fields; x200) was counted and averaged. Each group consisted of 3 or 4 Matrigel plugs.

The results showed that the number of blood vessels in the case of the Matrigel added with 5 μM of the fas-1 domain was significantly smaller than that in the control (see FIG. A of 9a). In the case of the Matrigel added with 10 μM of the fas-1 domain, angiogenesis was perfectly inhibited. Also, Matrigel was cut and its section was observed under a microscope, the results were the same as described above (see B and C of FIG. 9b). An in vivo angiogenesis inhibitory effect in the case of treatment with 5 μM of the fas-1 domain was significantly higher than that in the case of treatment with 500 μM of the YH motif-containing peptide fragment of 18 amino acids present in the fas-1 domain, in which the angiogenesis inhibitory effect of the peptide fragment had been confirmed previously by the present inventors (Nam, J. O. et al., J. Biol. Chem., 278:25902-25909, 2003).

The above results confirm that the fas-1 domain shows a more powerful angiogenesis inhibitory effect than that of the YH motif-containing peptide fragment of 18 amino acids present in the fas-1 domain.

### Example 10

### Analysis of Anticancer Effect of fas-1 Domain

10-1: Analysis of Proliferation of fas-1-Overexpressing Melanoma Cells

In order to examine whether the fas-1 domain is involved in the proliferation of melanoma cells, melanoma cells that overexpress the fas-1 domain were prepared and a test was performed using the cells.

cDNA (SEQ ID NO: 65) encoding the fourth fas-1 domain of βig-h3 was cloned into the EcoRI/XhoI sites of a pLNCX retrovirus vector (Clontech Lab. Inc., USA). The resulting recombinant retrovirus vector was named “fas-1/pLNCX”. Packaging cell PT67 (Clontech Lab. Inc., USA) were transfected with the fas-1/pLNCX vector using lipofectamine. The transformed cells were incubated at 37° C. for 48 hours. After completion of the incubation, virus particles present in the culture medium were collected, and filtered one time through a 0.45-μm filter. The filtered virus particles were added to a culture medium of melanoma cells (B16F10), and then allowed to react at 37° C. for 6 hours. The culture medium was replaced with fresh medium, and the transfected cells were screened several times in the presence of 1 mg/ml of G418. Western blot analysis using a βig-h3 antibody was performed to confirm that the fas-1 domain is overexpressed in the screened transfected cells. As a result, fas-1-overexpressing melanoma cells (hereinafter, referred to as “fas-1/B16F10”) were prepared. As a control, melanoma cells (pLNCX/B16F10) that overexpress an empty vector (pLNCX) containing no fas-1 cDNA were used.

Each of the fas-1-overexpressing melanoma cells (fas-1/B16F10) and the control cells (pLNCX/B16F10) was suspended in medium at a density of 3×10⁴ cells/ml, and 0.1 ml of the cell suspension was added to each well of a 96-well plate. The cells were incubated at 37° C. for 48-72 hours. 50 μl of 0.5 mg/ml thiazolyl blue (MTT; Sigma) solution was added to each well and allowed to react at 37° C. for 4 hours. After completion of the reaction, the medium was removed, and each well was added with DMSO and left to stand at room temperature for a few minutes. Thereafter, the absorbance was measured at 570 nm in a Bio-Rad model 550 microplate reader.

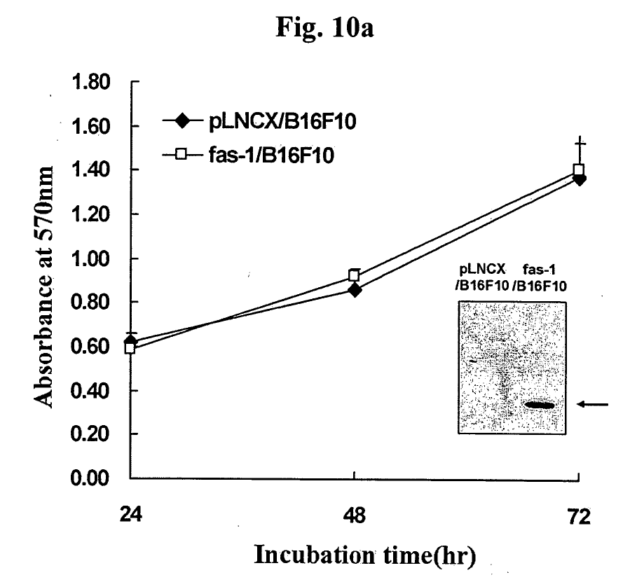

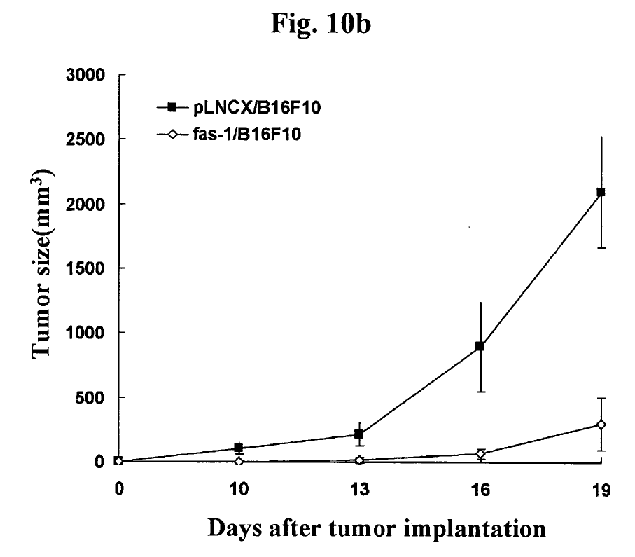

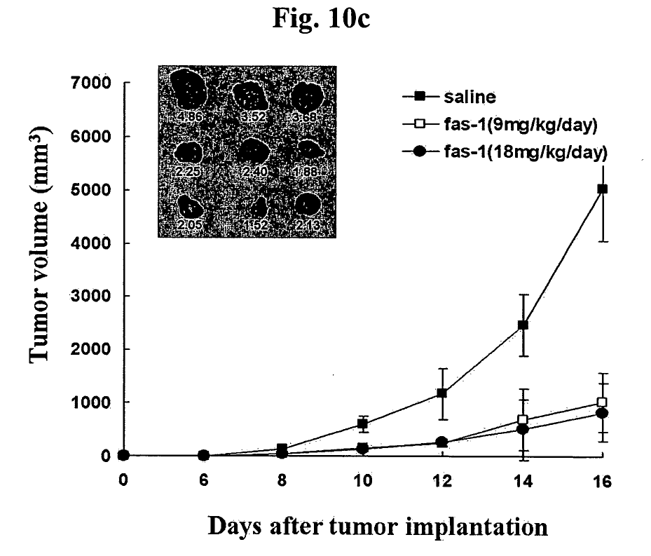

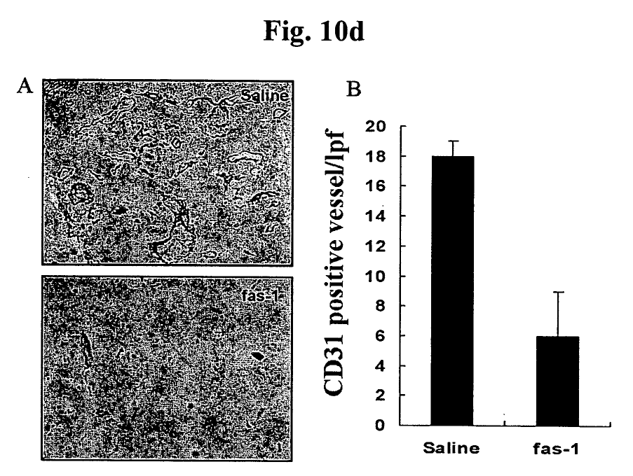

The results showed that, as shown in FIG. 10a, there was no difference in the proliferation between the fas-1/B16F10 cells and the pLNCX/B16F10 cells. This confirms that the fas-1 domain has no effect on the proliferation of melanoma cells.

10-2: Analysis of Growth of Tumors Derived from fas-1-Overexpressing Melanoma Cells

In present invention, whether the fas-1 domain influences the growth of tumors derived from the fas-1-overexpressing melanoma cells prepared in Example 10-1 above was tested in vivo.

Each of the fas-1-overexpressing melanoma cells (fas-1/B16F10) and the control cells (pLNCX/B16F10) was suspended in medium at a density of 1×10⁷ cells/ml. 0.1 ml of each of the cell suspensions was injected subcutaneously to 5-6 week old mice (Hyochang scientific company, Korea) to make a tumor model. Since the diameter of the tumor reached about 3 mm, the body weight and tumor size of the mice were checked at intervals of three days. The longest diameter and the shortest diameter of the tumor were measured with slide calipers, and the measured values were substituted in the following equation (1) to calculate the volume of the tumor.

Volume of tumor=a×b²/2 [Equation 1]

Wherein, ‘a’ is the shortest diameter of the tumor, and ‘b’ is the longest diameter of the tumor.

The results showed that, as shown in FIG. 10b, the size of the tumor derived from the pLNCX/B16F10 cells was increased rapidly with passage of time from 13 days after production of the tumor model. On the other hand, the tumor derived from the fas-1/B16F10 cells showed little or no increase in size even at 13 days after production of the tumor model, and then, the increase of tumor growth with the passage of time was not observed.

Such results confirm that the fas-1 domain does not affect the in vitro proliferation of melanoma cells, but it inhibits the in vivo growth of tumors derived from the melanoma cells.

10-3: Test of Tumor Growth Inhibitory Effect of fas-1 Domain

To further confirm the inhibitory effect of the fas-1 domain against tumor growth, the present inventors performed a test using BALB/c nude mice.

Melanoma cells B16F10 were suspended in medium at a density of 1×10⁷ cells/ml. 0.1 ml of the cell suspension was injected subcutaneously to 5-6 week old BALB/c nude mouse (purchased from Jung-Ang Animal Inc. Ltd., Korea) to make a subcutaneous tumor model. When the volume of the tumor reached about 25 mm3, mice having similar tumor size were selected and divided into three groups. The fas-1 domain (βig-h3 D-IV) prepared in Example 1 above was injected into the abdominal cavity of the mice of each group at the amount of 9 mg/kg or 18 mg/kg one time a day. A control was injected with saline solution. Thereafter, the body weight and tumor size of the mice were checked at intervals of three days. The diameter of the tumor was measured with slide calipers, and the volume of the tumor was calculated according to the equation 1 above.

The results showed that, as shown in FIG. 10c, the tumor in the control group started to grow rapidly from 10 days after induction of the tumor from the melanoma cells. On the other hand, the tumor in the test group injected with the fas-1 domain showed little or no increase in size even at 10 days after tumor induction, and then, its growth rate with time was much lower than that of the control.

Meanwhile, the tumor was removed after a given time, photographed with a stereoscopic microscope, weighed, and then fixed in 4% paraformaldehyde. The fixed tumor was immersed in 30%, 20% and 10% sucrose solutions one after another for one hour each solution, frozen rapidly with liquid nitrogen spray, and then left to stand at 80° C. for two hours. The frozen tumor was sliced with a tissue microtome (Leica, Germany) to make tissue slides which were then dried at room temperature. Thereafter, the tissue slides were immuno-stained with CD31 (Pharmingen, San Diego, Calif.), an antibody that binds specifically to vascular endothelial cells. The immuno-stained slides were observed under a microscope, and the number of blood vessels from 3-5 LPF (low power fields; x100) was counted and averaged. Each group consisted of 5 or 6 members.

The results showed that, as shown in FIG. 10d, the number of blood vessels stained with the antibody in the test group treated with the fas-1 domain was significantly smaller than that of the control. This suggests that fas-1 domain inhibits the growth of tumors by inhibiting angiogenesis.

### Example 11

### Assay of Inhibition of Endothelial Tube Formation by Various fas-1 Domains

To examine that the angiogenesis inhibitory effect of the fas-1 domain is either limited only to the fourth fas-1 domain of βig-h3 or the general effect of all the fas-1 domains of other proteins, the present inventors assayed the effects of the second fas-1 domain of human βig-h3 (SEQ ID NO: 3) and the third and seventh fas-1 domains of stabilin-II (SEQ ID NO: 45 and SEQ ID NO: 49) on endothelial tube formation.

For this purpose, the second fas-1 domain of βig-h3 (βig-h3 D-II) was prepared in the same manner as in Example 1-1 above. Since each domain of stabilin-II is insoluble, it was prepared in the form of soluble protein by cloning into the BamHI/XhoI sites of a Nus-conjugated pET43.1 vector (Novagen, USA). The prepared recombinant proteins were named “Nus-fas3” and “Nus-fas7”, respectively. A control to the second fas-1 domain of βig-h3 was treated with BSA, and a control to the fas-1 domain of stabilin-II was treated with a Nus protein. Thereafter, an endothelial tube formation assay was performed in the same manner as in Example 9-1.

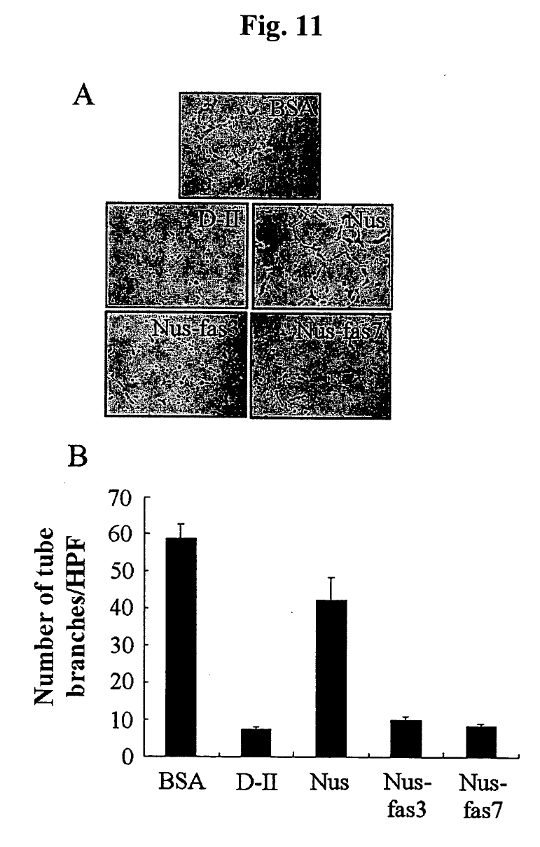

The results showed that, as shown in FIG. 11, the formation of endothelial tubes was not substantially inhibited by BSA or NUS, whereas the formation of endothelial cells in the test groups treated with βig-h3 D-II, Nus-fas3 or Nus-fas7 was completely inhibited.

The above results suggest that the inhibition of endothelial cell formation by the fas-1 domain is not limited only to the fourth domain of βig-h3 but the general effect of all the fas-1 domains of other proteins.

### Example 12

### Examination of Signal Transduction Pathway Related to Inhibition of Endothelial Cell Proliferation and Migration by fas-1 Domain

To examine the inhibitory mechanism of the fas-1 domain against endothelial cell proliferation and migration, the amount and activity of enzymes related to a FAK-Raf-ERK/AKT signal transduction pathway that is a typical signal transduction pathway in cells was determined by Western blot analysis on HUVECs treated with the fas-1 domain. Since the signal transduction-related proteins undergo phosphorylation during their activation process, the amount of phosphorylated proteins can be measured to predict their activity.

HUVECs were spread onto a 100-mm plate with 10% FBS-containing M199 medium, and incubated in an incubator at 37° C. for 12 hours. Following this, the culture medium was replaced with fresh medium containing 0.1% FBS and then further incubated for 24 hours. After removing the medium, the cells were washed one time with PBS buffer. The cells were detached from the plate by treatment with Trypsin-EDTA solution. The cells were suspended in 10% FBS-containing medium. Fas-1 protein was added to the cell suspension at final concentration of 20 μM. A control was added with PBS buffer. Thereafter, 3 ml of the cell mixture was taken and spread onto a plate coated with collagen. After 30 minutes, 60 minutes and 90 minutes, respectively, the medium was removed from the plate, and the cells were washed one time with PBS buffer. The cells were added with 200 μl of cell lysis buffer and left to stand on ice. The cell lysate was centrifuged at 12,000 rpm for 10 minutes at 4° C., and the supernatant containing a soluble protein was collected. Next, to quantify the concentration of the protein, a Bradford assay (BioRad, Hercules, Calif.) was performed using BSA as the standard protein. 30 μg of each of the sample proteins was electrophoresed on 10% SDS-polyacrylamide gel. The protein on the gel was transferred onto a nitrocellulose membrane, using electrophoresis. The protein transferred to the membrane was reacted with 5% skimmed milk for one hour to block nonspecific protein binding. Following this, it was reacted with the following various antibodies to signal transduction proteins: FAK (BD Science, Franklin Lakes, N.J., USA), pFAK (phosphorylated FAK; Santa Cruz Co., CA, USA), ERK (Sanca Cruz Co., CA, USA), pERK (phosphorylated ERK; Santa Cruz Co., CA, USA), AKT (Cell Signaling Technology Co., Beverly, Mass.), pAKT (phosphorylated AKT; Cell Signaling Technology Co., Beverly, Mass.), Raf 1 (Santa Cruz Co., CA, USA) and pRaf1 (phosphorylated Raf1, Santa Cruz Co., CA, USA). The antibodies had been diluted in TBST (50 mM Tris-HCl, pH 7.6, 150 mM NaCl, 0.1% Tween 20) before use. The binding reaction with the antibodies was performed for at least 12 hours in a refrigeration condition. After completion of the reaction, the membrane was washed three times with TBST solution. Thereafter, the membrane was added with HRP-conjugated secondary antibodies (HRP-conjugated-anti-mouse IgG and HRP-conjugated-anti-rabbit IgG; Santa Cruz Co., CA, USA), and a binding reaction with the secondary antibodies was performed at room temperature for one hour. Next, the membrane was washed three times, and then added with 1 ml of ECL as chemiluminescent solution, so that sites where antigen-antibody reaction occurred were visualized by fluorescence. The membrane was exposed to an X-ray film.

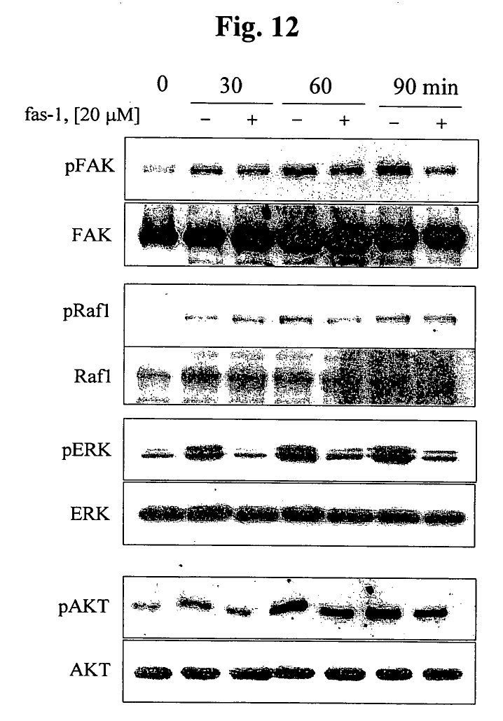

The amounts of the signal transduction-related proteins which had been phosphorylated as described above were analyzed for comparison. The results showed that, as shown in FIG. 12, in the case where HUVECs were attached to the collagen-coated plate, the phosphorylation of FAK, Raf1, ERK and AKT that are main enzymes related to a signal transduction pathway was increased in a time-dependent manner. On the other hand, in the case where the cells were treated with the fas-1 protein, the phosphorylation of the enzymes was not greatly increased or rather reduced, as compared to that of the control.

More concretely, the phosphorylation of FAK in the control group started to increase from 30 minutes, and further increased at 60 minutes and 90 minutes. However, the phosphorylation of FAK in the test group treated with the fas-1 domain was reduced as compared to the control. Particularly at 90 minutes, a difference in phosphorylation between the two groups was clearer. Also, the phosphorylation of AKT and Raf1 in the control group started to increase from 30 minutes in a similar manner as in FAK. However, the phosphorylation of AKT and Raf1 in the test group treated with the fas-1 protein was greatly reduced as compared to that of the control. Meanwhile, the phosphorylation of ERK in the control group started to increase from 30 minutes, but ERK in the test group treated with the fas-1 protein showed no increase in phosphorylation until 90 minutes.

Furthermore, the intracellular expression level of each enzyme was examined, and the results showed that the absolute amounts of FAK, Raf1, ERK and AKT enzymes in the cells were not significantly different between the control group and the test group treated with the fas-1 protein. This suggests that an increase in the phosphorylation of the enzymes is not attributable to an increase in the amount of the enzymes in the cells. The above results confirm that the fas-1 domain of βig-h3 inhibits the proliferation and migration of human umbilical vein endothelial cells (HUVECs) by inhibiting the FAK-Raf-ERK/AKT signal transduction pathway of HUVECs.

### Application Example 1

### Cancer

Angiogenesis is an essential stage in the growth and metastasis of cancer cells (Weidner, N. et al., N. Engl. J. Med., 324:1-8, 1991). Tumors are supplied with nutrients and oxygen necessary for their growth and proliferation through new blood vessels, and also new blood vessels invaded by tumors provides an opportunity for cancer cells to enter the blood circulation, thereby causing the metastasis of the cancer cells (Folkman and Tyler, Cancer Invasion and Metastasis, Biologic mechanisms and Therapy (S. B. Day ed.), Raven press, New York, 94-103, 1977; Polyerini P. J. Critical Reviews in Oral Biology, 6(3):230-247, 1995). If angiogenesis does not occur, the tumors will remain in a resting state and will no longer grow (Folkman and Tyler, Cancer Invasion and Metastasis, Biologic mechanisms and Therapy (S. B. Day ed.), Raven press, New York, 94-103, 1977). However, as angiogenesis in cancer tissues develops, cancer cell metastasis toward other tissues occurs (Weidner, N. et al., N. Engl. J. Med., 324:1-8, 1991). The metastasis of cancer cells by blood flow rarely occurs through preexisting blood vessels but mainly occurs at sites where angiogenesis actively occurs. In other words, cancer cells flow out through the incomplete walls of blood vessels, or flows out through the basement membrane of blood vessel walls when the basement membrane is degraded by the action of protease, thereby causing systemic metastasis. In some cases of systemic metastasis, endothelial cells being proliferated cause cancer cells to directly migrate into new blood vessels, thereby causing systemic metastasis. Accordingly, the inventive composition for the inhibition of angiogenesis, which contains the fas-1-containing polypeptide as an active ingredient, has an excellent angiogenesis inhibitory effect, and thus, is highly effective in the prevention of metastasis and the treatment of various cancers.

### Application Example 2

### Arthritis

Arthritis is an autoimmune disorder. However, a chronic inflammation, which is formed in the synovial cavity between joints during the progression of arthritis, induces angiogenesis to destroy cartilages. Arthritis includes infectious arthritis, degenerative arthritis, rheumatoid arthritis, and arthritis caused by femoral head avascular necrosis, ankylosing spondylitis and congenital malformation. Regardless of the cause of arthritis, the chronic inflammation formed in the synovial cavity between joints during the progression of arthritis is known to induce angiogenesis. It is characterized in that new capillary vessels invade joint to cause damage to cartilages (Kocb A. E. et al., Arth. Rheum., 29:471-479, 1986; Stupack D. G. et al., J. Med. Biol. Rcs., 32:578-281, 1999; Koch A. E., Arthritis Rheum., 41:951-962, 1998). In this case, it has been reported that an inflammatory response, which occurs in several steps depending the kind of diseases to destroy cartilages, plays an important role in the progression of the disease, and the formation of angiogenesis into joints acts as an important pathological mechanism (Colville-Nash, P. R. et al., Ann. Rheum. Dis., 51, 919-925, 1992; Eisenstein, R., Pharmacol. Ther., 49:1-19, 1991). For the treatment of arthritis, it is preferred to inhibit pains and inflammations so as to reduce the destruction rate of joints or muscles and minimize loss of their function. Accordingly, the inventive composition for the inhibition of angiogenesis is highly effective in the prevention of arthritis progression and in the treatment of arthritis.

### Application Example 3

### Psoriasis

Psoriasis is a skin disease that involves papules and silver white scars. It is generally a chronic proliferative disorder whose deterioration and improvement are repeated. Also, its cause is not yet identified, but it is known that the formation of new blood cells on pathological lesions or non-lesions, and also the infiltration of immune cells, such as neutrophil, as a result of an increase in blood vessel permeability, play an important role in the deterioration of psoriasis (Bhushan, M. et al., Br. J. Dermatol., 141:1054-1060, 1999). In normal persons, keratinocytes are proliferated one time a month, but in patients with psoriasis, keratinocytes are proliferated one time a week. Since much blood is necessary for this frequent proliferation, angiogenesis will necessarily occur fast (Folkman J. J. Invest. Dermatol., 59:40-48, 1972). Accordingly, the inventive composition for the inhibition of angiogenesis is effective in the treatment of psoriasis.

### Application Example 4

### Diabetic Eye Diseases

Ophthalmic diseases by which several million persons each year in the world lose their sight are also caused by angiogenesis (Jeffrey M. I. et al., J. Clin. Invest., 103:1231-1236, 1999; Stupack D. G. et al., J. Med. Biol. Rcs., 32:578-281, 1999). Typical examples of the ophthalmic diseases include age-related macular degeneration (AMD), diabetic retinopathy, retinopathy of prematurity, neovascular glaucoma, and corneal diseases caused by angiogenesis (Adamis A. P. et al., Angiogenesis, 3:9-14, 1999). Among them, the diabetic eye disease is one of main diabetic complications capable of causing loss of eyesight, and occurs in a patient with long diabetic duration regardless of the regulation of blood glucose. With a recent improvement in diabetic therapy, the lifespan of diabetic patients is extended while diabetic retinopathy shows a tendency to increase. Thus, the diabetic retinopathy is the leading cause of adult blindness in Western Europe and also Korea. The diabetic retinopathy develops due to the functional reduction of retinal circulation so that angiogenesis spreads along the internal surface and posterior hyaloid membrane of the retina while blood vessels invade the hyaloid, or bleeding occurs, resulting in blindness. Particularly, it has been reported that diabetic eye diseases, such as diabetic retinopathy, are caused by rapid progression of angiogenesis (Favard, C. et al., Diabetes, Metab., 22:268-273, 1996). Accordingly, the inventive composition for the inhibition of angiogenesis is highly effective in the prevention and treatment of diabetic eye diseases.

### Application Example 5

### Arterial Sclerosis

Sclerosis of the arteries means diseases where arterial walls become thicker to lose their elasticity. It is classified into three morphological categories, the most frequent and important category of which is atherosclerosis caused by the formation of atheroma in the arteries. The atheroma, which is formed of cholesterol and cholesterol ester and enclosed in a fibrous membrane, covers the tunica intima of the arteries while the lumen of arterial walls becomes narrower to block the blood flow of distal organs, thereby causing ischemic injury to the organs. If the atheroma is formed in the main artery, it then weakens the arterial walls to cause aneurysm, blood vessel disruption and thrombosis. In this case, it has been reported that the formation of new blood vessel within atheroma (angiogenesis) plays an important role in weakening the blood vessel walls (Hoshiga, M. et al., Circ. Res., 77:1129-1135, 1995; Kahlon, R. et al., Can. J. Cardiol., 8:60-64, 1992; George, S. J., Curr. Opin. Lipidol., 9:413-423, 1998). Accordingly, the inventive composition for the inhibition of angiogenesis is highly effective in the prevention of severe complications that can be caused by arterial sclerosis.

### Application Example 6

### Inflammation

Inflammation, which is a response of a living tissue to injury, can be caused by various factors, such as infection and trauma, but show substantially similar changes regardless of its cause and response tissue. Such changes include an increase in blood flow, an increase in permeability of blood vessel walls, and the infiltration of white blood cells, in which all the changes are known to be caused by angiogenesis (Jackson, J. R. et al., FASEB, J., 11:457-465, 1997). Although inflammation is a repairing mechanism of injury and thus not a harmful response, it can cause the injury and deformation of tissues when it excessively occurs or inappropriately occurs as in autoimmune diseases. In regulating such an excessive or inappropriate inflammatory response, the inventive composition for the inhibition of angiogenesis is effective.

### INDUSTRIAL APPLICABILITY

As described above, the polypeptide according to the present invention has an inhibitory effect on the adhesion, migration and proliferation of endothelial cells, as a result of interaction with the αvβ3 integrin of endothelial cells. Also, by such interaction with the αvβ3 integrin, the inventive polypeptide induces the apoptosis of endothelial cells and shows a powerful inhibitory effect on angiogenesis. Accordingly, the inventive polypeptide is useful for the inhibition of the adhesion, migration and/or proliferation of endothelial cells, and/or for the inhibition of angiogenesis. In addition, it is useful for the treatment or prevention of various angiogenesis-related diseases.

What is claimed is:

1. A method for inhibiting the adhesion, migration and/or proliferation of endothelial cells, which comprises administering to a subject in need thereof an effective amount of an isolated polypeptide comprising a fas-1 domain.

2. The method of claim 1, wherein the fas-1 domain is derived from mammals.

3. The method of claim 1, wherein the mammals are selected from the group consisting of human beings, rats and mice.

4. The method of claim 1, wherein the fas-1 domain is derived from any one selected from Dig-h3, periostin, stabilin-I and stabilin-II proteins.

5. The method of claim 1, wherein the polypeptide comprises an amino acid sequence selected from the group consisting of SEQ ID NO: 2 to SEQ ID NO: 65.

6. The method of claim 5, wherein the polypeptide comprises an amino acid sequence selected from the group consisting of SEQ ID NO: 2 to SEQ ID NO: 5, SEQ ID NO: 45, SEQ ID NO: 49, and SEQ ID NO: 61 to SEQ ID NO: 64.

7. A method for inducing the apoptosis of endothelial cells, which comprises administering to a subject in need thereof an effective amount of an isolated polypeptide comprising a fas-1 domain.

8. A method for inhibiting angiogenesis, which comprises administering to a subject in need thereof an effective amount of an isolated polypeptide comprising a fas-1 domain.

9. A method for the treatment or prevention of angiogenesis-related diseases, which comprises administering to a subject in need thereof an effective amount of an isolated polypeptide comprising a fas-1 domain.

10. The method of claim 9, wherein the angiogenesis-related diseases are selected from the group consisting of cancer, vascular malformation, arteriosclerosis, vascular adhesions, edematous sclerosis, corneal graft neovascularization, neovascular glaucoma, diabetic retinopathy, pterygium, retinal degeneration, retrolental fibroplasia, granular conjunctivitis, rheumatoid arthritis, systemic Lupus erythematosus, thyroiditis, psoriasis, capillarectasia, pyogenic granuloma, seborrheic dermatitis and acne.

11. A pharmaceutical composition for the inhibition of angiogenesis, which comprises as an active ingredient an isolated polypeptide comprising a fas-1 domain.

12. A pharmaceutical composition for the treatment or prevention of angiogenesis-related diseases, which comprises as an active ingredient an isolated polypeptide comprising a fas-1 domain.

13. Use of an isolated polypeptide comprising a fas-1 domain, for the preparation of a pharmaceutical agent of inhibiting the adhesion, migration and/or proliferation of endothelial cells.

14. Use of an isolated polypeptide comprising a fas-1 domain, for the preparation of a pharmaceutical agent of inducing the apoptosis of endothelial cells.

15. Use of an isolated polypeptide comprising a fas-1 domain, for the preparation of an angiogenesis-inhibiting agent.

16. Use of an isolated polypeptide comprising a fas-1 domain, for the preparation of a therapeutic or preventive agent for angiogenesis-related diseases.
